(19) United States
(12) Patent Application Publication
(10) Pub. No.: US 2015/0147340 A1
(43) Pub. Date: May 28, 2015

(54) THERAPEUTIC METHODS FOR PERITONEAL CARCINOMATOSIS

(71) Applicant: FibroGen, Inc., San Francisco, CA (US)

(72) Inventors: Goli Samimi, Bondi Junction (AU); Kim Moran-Jones, Parramatta (AU)

(21) Appl. No.: 14/389,315

(22) PCT Filed: Mar. 12, 2013
(86) PCT No.: PCT/US2013/030501

§ 371 Date: Sep. 29, 2014

Related U.S. Application Data
(60) Provisional application No. 61/617,849, filed on Mar. 30, 2012.

(51) Int. Cl. C07K 16/22; A61K 31/713; C12N 15/113; A61K 39/395

(52) U.S. Cl. CPC C07K 16/22; A61K 39/3955; A61K 31/713; C12N 15/1136; C07K 2317/76; A61K 2039/505
USPC 424/158.1; 514/44 A

(57) ABSTRACT

Described herein are methods and medicaments useful for treating peritoneal carcinomatosis by administering anti-CTGF agents, particularly anti-CTGF antibodies. Methods for prognosing individuals with perinoteal carcinomatosis are also provided. In one aspect, the present invention provides a method of treating a subject with peritoneal carcinomatosis, the method comprises the administration to the subject of an effective amount ohm anti-connective tissue growth factor (CTGF) agent, thereby treating the peritoneal carcinomatosis. In some embodiments, the peritoneal carcinomatosis results from a cancer selected from the group consisting of gall bladder cancer, bile duct cancer, liver cancer, colon cancer, cancer of the appendix, ovarian cancer, fallopian tube cancer, bladder cancer, pancreatic cancer, mesothelioma, rectal cancer, small bowel cancer and stomach cancer. In particular embodiments, the cancer is ovarian cancer. In further embodiments, the ovarian cancer is classified as serous, clear cell, mucinous or endometrioid.

### CROSS REFERENCE TO RELATED APPLICATIONS

This application claims the benefit under 35 U.S.C. §119(e) of U.S. Provisional Application 61/617,849 filed Mar. 30, 2012 and is hereby incorporated by reference in its entirety.

### FIELD OF THE INVENTION

The present invention relates to methods and medicaments useful for treating peritoneal carcinomatosis. Methods for prognosing individuals with peritoneal carcinomatosis are also provided.

### BACKGROUND OF THE INVENTION

Peritoneal carcinomatosis is metastastic disease within the peritoneal cavity that originates from primary cancers of the peritoneum, or more commonly, from cancers that originate in other organs or tissues. Peritoneal carcinomatosis is a terminal condition with a median survival time of 6 months. (Levine E A et al. Am Coll Surg. 2007; 204:943-53.) Numerous types of cancers metastasize to the peritoncal cavity including gynecologic cancers such as endometrial, fallopian tube, ovarian and uterine cancers; gastrointestinal cancers such as colorectal and stomach cancers; gall bladder, pancreatic cancer, liver cancer and breast cancer. The condition is particularly common in epitheal ovarian cancer patients, where about 75% to 85% of patients at the time of diagnosis have peritoneal carcinomatosis. (Ozols R F et al. Hoskins W J, Young R C, Markman M, Perez C A, Barakat R, Randall M. Gynecologic Oncology. 4th Ed Lippincott Williams & Wilkins; Philadelphia, Pa.: 2005. Epithelial ovarian cancer, p. 916.) Other cancers that frequently have peritoneal involvement include gastric cancer where up to 30% of the patients have peritoneal carcinomatosis at time of diagnosis (Cabourne E. et al. J Surg Res 2010; 164:e265-e272) and colorectal cancer, where over 15% of patients have peritoneal carcinomatosis at the time of diagnosis (Chang G J, Lambert L A. Ann Surg Oncol 2008; 15:2993-95).

The dire prognosis faced by patients with peritoneal carcinomatosis requires the development of new treatment methods and agents for effectively treating peritoneal carcinomatosis. The present invention meets these needs by providing agents that inhibit connective tissue growth factor (CTGF) expression or activity and methods for administering these agents.

### SUMMARY OF THE INVENTION

The present invention provides methods and anti-CTGF agents that are useful in the treatment of peritoneal carcinomatosis. In one aspect, the present invention provides a method of treating a subject with peritoneal carcinomatosis, the method comprises the administration to the subject of an effective amount of an anti-connective tissue growth factor (CTGF) agent, thereby treating the peritoneal carcinomatosis. In some embodiments, the peritoneal carcinomatosis results from a cancer selected from the group consisting of gall bladder cancer, bile duct cancer, liver cancer, colon cancer, cancer of the appendix, ovarian cancer, fallopian tube cancer, bladder cancer, pancreatic cancer, mesothelioma, rectal cancer, small bowel cancer and stomach cancer. In particular embodiments, the cancer is ovarian cancer. In further embodiments, the ovarian cancer is classified as serous, clear cell, mucinous or endometrioid.

In some embodiments the anti-CTGF agent is an anti-CTGF antibody, antibody fragment or antibody mimetic. In further embodiments, the CTGF agent is an anti-CTGF antibody. In specific embodiments, the anti-CTGF antibody is identical to the antibody produced by the cell line identified by ATCC Accession No. PTA-6006.

In other embodiments, the anti-CTGF agent is an anti-CTGF oligonucleotide. In further embodiments, the anti-CTGF oligonucleotide is an antisense oligonucleotide, siRNA, ribozyme or shRNA.

In some embodiments, the anti-CTGF agent is administered interperitoneally. In further embodiments, the anti-CTGF agent is administered as a neoadjuvant. In other embodiments, the treatment method further comprises the administration of another therapeutic modality selected from the group consisting of chemotherapy, immunotherapy, gene therapy, surgery, radiotherapy, or hyperthermia. In specific embodiments, the chemotherapy is hyperthermic interperitoneal chemotherapy. In other embodiments, the surgery is cytoreductive surgery.

In another aspect, the present invention provides a method for inhibiting cancer cell adherence to or growth on the peritoneal membrane of a subject, the method comprises the administration of a therapeutically effective amount of an anti-CTGF agent, thereby inhibiting cancer cell adherence or growth on the peritoneal membrane. In some embodiments, the subject has peritoneal carcinomatosis.

In one aspect of the invention, a method is provided for prognosing a subject with ovarian cancer, the method comprises determining the percentage of tumor-associated fibroblasts in an ovarian carcinoma sample obtained from the subject that are positive for CTGF expression, and prognosing the subject based on the percentage of CTGF positive tumor-associated fibroblasts compared to a reference percentage. In some embodiments, CTGF expression is CTGF mRNA expression. In other embodiments, CTGF expression is CTGF protein expression. In further embodiments, the prognosis is an aggressive form of ovarian cancer or a lower overall survival rate if the percentage of CTGF positive tumor-associated fibroblasts is greater than the reference percentage.

These and other embodiments of the present invention will readily occur to those of skill in the art in light of the disclosure herein, and all such embodiments are specifically contemplated. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

FIG. 1 shows an unsupervised hierarchical clustering analysis of the 9,741 probe sets passing filtering criteria using Euclidean distance with average linkage. Clustering can discriminate between normal ovarian fibroblasts and tumor-associated fibroblast samples.

FIG. 2 illustrates the results of a validation study where nine genes shown to be differentially expressed between normal and high-grade serous ovarian cancer (HGSOC)-associated fibroblasts (tumor-associated fibroblasts) by microarray analysis were compared by quantitative real-time PCR (qRT-PCR). The qRT-PCR data confirmed the results of the microarray analysis. These data were calculated using the 2^{-CTΔΔ} method and p-values for expression differences were calculated between ovarian tumor-associated fibroblasts and normal ovarian fibroblasts. *p-value<10⁻², **p-value<10⁻⁴, ***p-value<10⁻⁶

FIG. 3 illustrates the difference in CTGF expression obtained by microarray analysis between HGSOC-associated fibroblasts (white bars) and matched tumor epithelial cells obtained from the same individuals (black bars). The difference in CTGF expression was highly significant (p-value<10⁻⁷). In contrast, CTGF expression did not differ between normal ovary epithelial cells and ovarian fibroblasts (data not shown).

FIG. 4 illustrates TGF-β-stimulated secretion of CTGF (ng/μg total cellular protein) into media by normal ovarian fibroblasts (NF), ovarian cancer-associated fibroblasts (CAF) and OVCAR3 ovarian cancer cells of epithelial origin. Cells were placed in serum-free media and either untreated (white bars) or treated with 10 ng/ml TGF-β (black bars). After 24 hours, the media was collected and tested for CTGF concentration. Both types of fibroblasts secrete significantly higher basal and TGF-β-stimulated levels of CTGF in comparison with OVCAR3 cells, a proxy for epithelial cells. (p<0.05)

FIG. 5 illustrates CTGF-stimulated ovarian cancer cell motility. Three ovarian cancer cell lines A224 (black bars), OVCAR3 (white bars) and SKOV3 (gray bar) were exposed to increasing concentrations of recombinant human CTGF (rhCTGF) for six hours. A dose response is seen with r=0.91 for A224 cells, r=0.68 for OVCAR3 cells and r=0.78 for SKOV3 cells.

FIG. 6 demonstrates that treatment with an anti-CTGF antibody (CLN1) blocks CTGF-stimulated migration. Untreated cells (white bars); cells treated with 5 μg/ml rhCTGF (black bars); cells treated with 5 μg/ml rhCTGF and 100 μg/ml CLN1 (light gray bars); and cells treated with with 5 μg/ml rhCTGF and 100 μg/ml IgG (dark gray bar). Each bar represents the mean of triplicate wells±SD. *p-value<0.008, **p-value<0.004, ***p-value<0.02, ****p-value<0.003

FIG. 7 demonstrates that stably transfected OVCAR3 cells overexpressing CTGF exhibit anchorage independent growth in soft agar. In contrast, stably transfected OVCAR3 cells transfected with the empty vector exhibited minimal growth. Cells were stained with nitroblue tetrazolium after 10-14 days of growth and colonies between 100-2000 microns were counted. Each bar represents the mean of triplicate wells±SD. *p-value<0.0001.

FIG. 8 illustrates the ability of rhCTGF to increase ea-vivo peritoneal tissue adhesion of OVCAR3 cells and also the ability of an anti-CTGF antibody to block the CTGF-stimulated increase in adhesion. OVCAR3 cells untreated; treated with 5 μg/ml rhCTGF; treated with 5 μg/ml rhCTGF and 50 μg/ml CLN1 or treated with 5 μg/ml rhCTGF and 125 μg/ml IgG, were placed on peritoneal tissue for two hours. After two hours, the peritoneal tissue was washed and the number of cells attached to the tissue was counted. Each bar represents the average of 3 fields in 3 independent experiments±SD. CTGF significantly increases the number of ovarian cancer cells that attach to the peritoneal tissue, *p-value<2×10⁻⁶, while anti-CTGF antibody blocks the effect of CTGF, **p-value<2×10⁻⁸.

FIG. 9 illustrates the relationship between tumor-associated fibroblast CTGF expression and survival in patients with serous ovarian cancer. Patients whose tumor-associated fibroblasts expressed high levels of CTGF (score 2 or 3) survived for a median time of 19 months compared to a median survival time of 24 months for patients whose tumor-associated fibroblasts expressed low levels of CTGF (score 0 or 1).

FIG. 10 illustrates the relationship between tumor-associated fibroblast CTGF expression and survival of patients with serous ovarian cancer. Patients with tumor-associated fibroblasts that had ≦90% CTGF expression survived for a median of 38.0 months versus a median survival of 9.0 months for patients with tumor-associated fibroblasts that had >90% CTGF expression (n=88; p-value=0.0006).

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It should be noted that as used herein and in the appended claims, the singular forms “a,” “an,” and “the” include plural references unless context clearly dictates otherwise. Thus, for example, a reference to “an anti-CTGF antibody” may include a plurality of such antibodies.

As used herein the term “about” refers to ±10% of the numerical value of the number with which it is being used. Therefore, about 50% means in the range of 45%-55%.

As used herein, the term “subject,” “individual,” and “patient” are used interchangeably to refer to a mammal. In a preferred embodiment, the mammal is a primate, and more preferably a human being.

The term “peritoneal carcinomatosis,” as used herein, refers to the neoplastic involvement of the peritoneum, typically seen as wide-spread seeding or growth of tumor masses or metastases. Peritoneal carcinomatosis can result from primary or secondary carcinomas. Primary peritoneal carcinomas arise from peritoneum cells and since the mesothelium of the peritoneum and the germinal epithelium of the ovary have the same embryologic origin, the peritoneum retains the multipotentiality allowing for the development of a primary carcinoma that can then spread within the peritoneal cavity. Primary carcinomas that cause peritoneal carcinomatosis and are contemplated for treatment using the disclosed methods and agents include malignant mesothelioma, benign papillary mesothelioma, desmoplastic small round cell tumors, peritoneal angiosarcoma, leiomyomatosis peritonealis disseminata (LPD), and peritoneal hemangiomatosis. Additionally, ovarian cancer arising in women after bilateral oophorectomy is included as a primary peritoneal cancer that can result in peritoneal catcinomatosis.

Much more commonly, peritoneal carcinomatosis results from a cancer that arises in an anatonomically separate location and later metastasizes to the peritoneal cavity. Numerous cancers can produce peritoneal carcinomatosis including cancers of the endometrium, fallopian tubes, ovaries, uterus, colon, rectum, small bowel, gall bladder, bile duct, appendix, stomach, pancreas, liver and breast. In some embodiments, the cancer that produces peritoneal carcinomatosis is not pancreatic cancer.

In some embodiments, the peritoneal carcinomatosis results from ovarian cancer. As used herein, “ovarian cancer” or “ovarian tumor” includes any tumor, cell mass or micrometastasis derived from, or originating from cells of the ovary. This includes tumors originating from the epithelial cell layer (serous) of the ovary. Ovarian cancer further includes secondary cancers of ovarian origin and further includes recurrent or refractory disease.

In further embodiments, the peritoneal carcinomatosis is pseudomyxoma peritonei, the peritoneal dissemination of an appendiceal mucinous epithelial neoplasm, a relatively slow growing cancer that is characterized by the excessive production of mucinous ascites. (Smeenk R M, et al. Pseudomyxoma peritonei. Cancer Treat Rev 2007, 33:138-145).

An “advanced” cancer, as used herein, refers to a cancer that has spread outside of the tissue or organ of origin, either by local invasion, lymph node involvement, or by metastasis. Advanced cancers comprise peritoneal carcinomatosis including peritoneal carcinomatosis from primary cancers of the peritoneum.

A “refractory” cancer, as used herein, refers to a cancer that has progressed even though an anti-cancer therapy, such as a chemotherapy agent, was being administered to the patient. An example of a refractory cancer is ovarian cancer that does not respond or continues to progress while the patient is administered standard chemotherapy, i.e., platinum-based chemotherapy.

A “recurrent” cancer, as used herein, refers to a cancer that has regrown, either at the site of origin or at a distant site, following an initial response to therapy. Recurrent cancers include cancers that recur in the peritoneal cavity following treatment such as ovarian cancer, colon cancer, pancreatic cancer and stomach cancer. Recurrent cancers in the peritoneal cavity usually result in peritoneal carcinomatosis.

As used herein, the terms “cancer-associated fibroblasts,” “tumor-associated fibroblasts” and “tumor stromal fibroblasts” refer to fibroblasts and myofibroblasts that are components of tumor stroma including tumor stroma from serous ovarian carcinoma. High grade serous ovarian cancer (HGSOC)-associated fibroblasts are a subset of cancer-associated fibroblasts.

As used herein, the terms “treating,” “treatment” and “therapy” mean to administer an anti-CTGF agent to a subject with peritoneal carcinomatosis, including subjects with disease at the original site of cancer occurrence, distant metastases and occult disease. The peritoneal carcinomatosis can be newly diagnosed, refractory or recurrent disease. The administration of an anti-CTGF agent to the subject can have the effect of, but is not limited to, preventing, reducing or inhibiting the adherence of cancer cells to the peritoneal membrane; preventing, reducing or inhibiting the growth rate of cancer cells on the peritoneal membrane; reducing or inhibiting the motility and/or invasiveness of cancer cells within the peritoneal cavity; inducing apoptosis; sensitizing cancer cells to chemotherapy drugs, biologic agents and/or radiation; increasing the effectiveness of another therapeutic modality, such as chemotherapy, in an additive or synergistic manner.

As used herein, “prognosing” or “prognosis” refers to predicting the probable clinical course and outcome of an ovarian cancer patient. The prognosis can include the presence of aggressive disease, the likelihood of tumor response or sensitivity to a particular treatment, the likelihood of recurrence, and an estimate of patient survival. Prognosing can also be used to segregate patients into a poor survival group or a good survival group associated with a disease subtype which is reflected by the extent of CTGF expression (mRNA or protein) in the tumor-associated fibroblasts.

“Connective Tissue Growth Factor (CTGF)” is a 36 kD, cysteine-rich, heparin binding secreted glycoprotein originally isolated from the culture media of human umbilical vein endothelial cells. (Bradham et al. (1991) J Cell Biol 114:1285-1294; Grotendorst and Bradham, U.S. Pat. No. 5,408,040.) CTGF belongs to the CCN (CTGF, Cyr61, Nov) family of proteins, which includes the serum-induced immediate early gene product Cyr61, the putative oncogene Nov, and the Wnt-inducible secreted proteins (WISP)-1, -2, and -3. (See, e.g., O'Brian et al. (1990) Mol Cell Biol 10:3569-3577; Joliot et al. (1992) Mol Cell Biol 12:10-21; Ryseck et al. (1991) Cell Growth and Diff 2:225-233; Simmons et al. (1989) Proc. Natl. Acad. Sci. USA 86:1178-1182; Pennica et al. (1998) Proc Natl Acad Sci USA, 95:14717-14722; and Zhang et al. (1998) Mol Cell Biol 18.6131-6141.) CCN proteins are characterized by conservation of 38 cysteine residues that constitute over 10% of the total amino acid content and give rise to a modular structure with N- and C-terminal domains. The modular structure of CTGF includes conserved motifs for insulin-like growth factor binding proteins (IGF-BP) and von Willebrand's factor (VWC) in the N-terminal domain, and thrombospondin (TSP1) and a cysteine-knot motif in the C-terminal domain.

Although the present invention demonstrates that agents that inhibit CTGF activity can reduce or inhibit CTGF-induced anchorage-independent proliferation, cell migration and adhesion to the peritoneal membrane, the invention specifically contemplates inhibiting the expression or activity of other CCN family members for the treatment of peritoneal carcinomatosis, particularly Cyr61.

CTGF expression is induced by various factors including TGF-β family members, e.g., TGF-β1, activin, etc.; thrombin, vascular endothelial growth factor (VEGF), endothelin and angiotensin II. (Franklin (1997) Int J Biochem Cell Biol 29:79-89; Wunderlich (2000) Graefes Arch Clin Exp Ophthalmol 238:910-915; Denton and Abraham (2001) Curr Opin Rheumatol 13:505-511; and Riewald (2001) Blood 97:3109-3116; Xu et al. (2004) J Biol Chem 279.23098-23103.) Therefore, in some embodiments, the present invention is directed to combination treatment with anti-CTGF agents and agents that antagonize or inhibit the activity or expression of TGF-β family members, VEGF, endothelin and angiotensin 1.

A “package insert” is used to refer to instructions customarily included in commercial packages of therapeutic products that contain information about the indications, usage, dosage, administration, contraindications, other therapeutic products to be combined with the packaged product, and/or warnings concerning the use of such therapeutic products, etc.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R, and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### Antibodies

The term “antibody” is used in the broadest sense and specifically covers monoclonal antibodies (including full length monoclonal antibodies), polyclonal antibodies, multispecific antibodies (e.g., bispecific antibodies), antibody fragments, so long as they exhibit the desired biological activity, and antibody mimetics.

Anti-CTGF antibodies (i.e., antibodies that specifically bind CTGF or fragments of CTGF) can be prepared using intact CTGF polypeptides, fragments of CTGF or small polypeptides or oligopeptides as the immunizing antigen. The polypeptide or oligopeptide used to immunize an animal (e.g., a mouse, rat, rabbit, chicken, turkey, goat, etc.) can be derived, inter alia, from proteolysis of the CTGF protein, the translation of CTGF mRNA, or synthesized chemically, and can be conjugated to a carrier protein if desired. Commonly used carriers chemically coupled to peptides include, for example, bovine serum albumin, thyroglobulin, and keyhole limpet hemocyanin (KLH). Other methods of selecting antibodies having desired specificities (e.g., phage display) are well known in the art.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Harlow et al., Antibodies: A Laboratory Manual, (Cold Spring Harbor Laboratory Press, 2nd ed. 1988); recombinant DNA methods (see, e.g., U.S. Pat. No. 4,816,567); phage-display technologies (see, e.g., Clackson et al., Nature, 352:624-628 (1991); Marks et al., J Mol Biol. 222: 581-597 (1992); and Lee et al., J Immunol Methods 284(1-2): 119-132(20041 and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc Nat. Aca Sci USA 90: 2551 (1993); U.S. Pat. Nos. 5,545,807; 5,545,806; 5,569,825; 5,625,126; 5,633,425; and 5,661,016.

Monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass (see, e.g., U.S. Pat. No. 4,816,567; and Morrison et al., Proc. Natl Acad Sci USA 81:6851-6855 (1984)).

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from a one or more hypervariable regions (HVRs) of the recipient are replaced by residues from one or more HVRs of a non-human species (donor antibody) such as mouse, rat, rabbit, or nonhuman primate having the desired specificity, affinity, and/or capacity. For further details, see, e.g., Jones et al., Nature 321:522-525 (1986); Riechmann et al., Nature 332:323-329 (1988); and U.S. Pat. Nos. 6,982,321 and 7,087,409.

A “human antibody” is one which possesses an amino acid sequence which corresponds to that of an antibody produced by a human and/or has been made using any of the techniques for making human antibodies (see e.g., Hoogenboom and Winter, J Mol Biol, 227:381 (1991); Marks et al., J Mol Biol, 222:581 (1991); Boerner et al., J Immunol, 147(1):86-95 (1991); Li et al., Proc Natl Acad Sci USA, 103:3557-3562 (2006) and U.S. Pat. Nos. 6,075,181 and 6,150,584).

The term “neutralizing antibody” as used herein refers to an antibody, preferably a monoclonal antibody, that is capable of substantially inhibiting or eliminating a biological activity of CTGF. Typically, a neutralizing antibody will inhibit binding of CTGF to a cofactor such as TGFβ, to a CTGF-specific receptor associated with a target cell, or to another biologic target.

A “naked antibody” for the purposes herein is an antibody that is not conjugated to a cytotoxic moiety or radiolabel. In some embodiments, the anti-CTGF antibody is a naked antibody.

The anti-CTGF antibodies disclosed herein bind specifically to CTGF. Anti-CTGF antibodies may be specific for CTGF endogenous to the species of the subject to be treated or may be cross-reactive with CTGF from one or more other species. In some embodiments, the antibody for use in the present methods is obtained from the same species as the subject in need. In other embodiments, the antibody is a chimeric antibody wherein the constant domains are obtained from the same species as the subject in need and the variable domains are obtained from another species. For example, in treating a human subject, the antibody for use in the present methods may be a chimeric antibody having constant domains that are human in origin and variable domains that are mouse in origin. In preferred embodiments, the antibody for use in the present methods binds specifically to the CTGF endogenous to the species of the subject in need. Thus, in certain embodiments, the antibody is a human or humanized antibody, particularly a monoclonal antibody, that specifically binds human CTGF, GenBank Accession No. NP_{—}001892.

Exemplary antibodies for use in the methods of the present invention are described, e.g., in U.S. Pat. No. 5,408,040; PCT/US1998/016423; PCT/US1999/029652 and International Publication No. WO 99/33878. In some embodiments, the anti-CTGF antibody for use in the methods is a monoclonal antibody. Preferably, the antibody is a neutralizing antibody. In particular embodiments, the antibody is an antibody described and claimed in U.S. Pat. Nos. 7,405,274 and 7,871,617. In some embodiments, the antibody has the amino acid sequence of the antibody produced by the cell line identified by ATCC Accession No. PTA-6006, i.e., it is identical to the antibody produced by this cell line. In other embodiments, the antibody binds to CTGF competitively with an antibody produced by the cell line identified by ATCC Accession No. PTA-6006. In further embodiments, the antibody binds to the same epitope as the antibody produced by ATCC Accession No. PTA-6006. A particular antibody for use in the present methods is CLN1 or mAb1, as described in U.S. Pat. No. 7,405,274 and U.S. patent application Ser. No. 12/148,922, or an antibody substantially equivalent thereto or derived therefrom.

As used herein, “specific binding” refers to the antibody binding to a predetermined antigen. Typically, the antibody binds the antigen with a dissociation constant (K_{D}) of 10⁻⁷ M or less, and binds to the predetermined antigen with a K_{D} that is at least 1.5-fold less, at least 2-fold less or at least 5-fold less than its K_{D} for binding to a non-specific antigen (e.g., bovine serum albumin or casein). The phrases “an antibody recognizing an antigen” and “an antibody specific for an antigen” are used interchangeably herein with the term “an antibody which specifically binds to an antigen.”

As referred to herein, the phrase “an antibody that specifically binds to CTGF” includes any antibody that binds to CTGF with high affinity. Affinity can be calculated from the following equation:

$$Affinity = {K}_{a} = \frac{[ Ab \cdot Ag ]}{[ Ab ] [ Ag ]} = \frac{1}{{K}_{d}}$$

where [Ab] is the concentration of the free antigen binding site on the antibody, [Ag] is the concentration of the free antigen, [Ab-Ag] is the concentration of occupied antigen binding sites, Ka is the association constant of the complex of antigen with antigen binding site, and Kd is the dissociation constant of the complex. A high-affinity antibody typically has an affinity at least on the order of 10⁸ M⁻¹, 10⁹ M⁻¹ or 10¹⁰ M⁻¹. In particular embodiments, an antibody for use in the present methods will have a binding affinity for CTGF between of 10⁸ M⁻¹ and 10¹⁰ M⁻¹, between 10⁸ M⁻¹ and 10⁹ M⁻¹ or between 10⁹ M⁻¹ and 10¹⁰ M⁻¹. In some embodiments, the high-affinity antibody has an affinity of about 10⁸ M⁻¹, 10⁹ M⁻¹ or 10¹⁰ M⁻¹. Anti-CTGF antibodies used in the present invention preferably have a K_{D} for CTGF of 10⁻⁸ M or less.

“Antibody fragments” comprise a functional fragment or portion of an intact antibody, preferably comprising an antigen binding region thereof. A functional fragment of an antibody will be a fragment with similar (not necessarily identical) specificity and affinity to the antibody from which it was derived. Non-limiting examples of antibody fragments include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody; and (v) an isolated complementarity determining region (CDR). Fab, F(ab′)₂, and Fv fragments can be produced through enzymatic digestion of whole antibodies, e.g., digestion with papain, to produce Fab fragments. Other non-limiting examples include engineered antibody fragments such as diabodies (Holliger P et al. Proc Natl Acad Sci USA. 1993, 90: 6444-6448); linear antibodies (Zapata et al. 1995 Protein Eng, 8(10):1057-1062); single-chain antibody molecules (Bird K D et al. Science, 1988, 242: 423-426); single domain antibodies, also known as nanobodies (Ghahoudi M A et al. FEBS Lett. 1997, 414: 521-526); domain antibodies (Ward E S et al. Nature. 1989, 341: 544-546); and multispecific antibodies formed from antibody fragments.

### Antibody Mimetics

Antibody mimetics are proteins, typically in the range of 3-25 kD that are designed to bind an antigen with high specificity and affinity like an antibody, but are structurally unrelated to antibodies. Frequently, antibody mimetics are based on a structural motif or scaffold that can be found as a single or repeated domain from a larger biomolecule. Examples of domain derived antibody mimetics included AdNectins that utilize the 10th fibronectin III domain (Lipov\dot{s}ek D. Protein Eng Des Sel, 2010, 243-9); Affibodies that utilize the Z domain of staphylococcal protein A (Nord K et al. Nat Biotechnol. 1997, 15: 772-777) and DARPins that utilize the consensus ankyrin repeat domain (Amstutz P. Protein big Des Sel. 2006, 19:219-229. Alternatively, antibody mimetics can also be based on substantially the entire structure of a smaller biomolecule, such as Anticalins that utilize the lipocalin structure (Beste G et al. Proc Natl Acad Sci USA. 1999, 5:1898-1903)

### Oligonucleotides

The terms “oligonucleotide” and “oligomeric nucleic acid” refer to oligomers or polymers of ribonucleic acid (RNA), deoxyribonucleic acid (DNA), mimetics or analogs of RNA or DNA, or combinations thereof in either single- or double-stranded form. Oligonucleotides are molecules formed by the covalent linkage of two or more nucleotides or their analogs. Unless specifically limited, the term encompasses nucleic acids containing analogues of natural nucleotides that have similar binding properties as the reference nucleic acid.

Oligonucleotides for use in the invention are linear molecules or are synthesized as linear molecules. In some embodiments, the oligonucleotides are antisense oligonucleotides and not small interfering RNAs (siRNAs). In further embodiments, the oligonucleotides of the invention are siRNAs and not antisense oligonucleotides. In other embodiments, the oligonucleotides of the invention are not ribozymes, external guide sequence (EGS) oligonucleotides (oligozymes), or other short catalytic RNAs.

The terms “complementary” and “complementarity” refer to conventional Watson-Crick base-pairing of nucleic acids. For example, in DNA complementarity, guanine forms a base pair with cytosine and adenine forms a base pair with thymine, whereas in RNA complementarity, guanine forms a base pair with cytosine, but adenine forms a base pair with uracil in place of thymine. An oligonucleotide is complementary to a RNA or DNA sequence when the nucleotides of the oligonucleotide are capable of forming hydrogen bonds with a sufficient number of nucleotides in the corresponding RNA or DNA sequence to allow the oligonucleotide to hybridize with the RNA or DNA sequence.

As used herein, the term “antisense oligonucleotide” refers to an oligomeric nucleic acid that is capable of hybridizing with its complementary target nucleic acid sequence resulting in the modulation of the normal function of the target nucleic acid sequence. In some embodiments, the modulation of function is the interference in function of DNA, typically resulting in decreased replication and/or transcription of a target DNA. In other embodiments, the modulation of function is the interference in function of RNA, typically resulting in impaired splicing of transcribed RNA (pre-mRNA) to yield mature mRNA species, reduced RNA stability, decreased translocation of the target mRNA to the site of protein translation and impaired translation of protein from mature mRNA. In other embodiments, the modulation of function is the reduction in cellular target mRNA (e.g., CTGF mRNA) number or cellular content of target mRNA (e.g., CTGF mRNA). In some embodiments, the modulation of function is the down-regulation or knockdown of gene expression. In other embodiments, the modulation of function is a reduction in protein expression or cellular protein content.

The terms “small interfering RNA” or “siRNA” refer to single- or double-stranded RNA molecules that induce the RNA interference pathway and act in concert with host proteins, e.g., RNA induced silencing complex (RISC) to degrade mRNA in a sequence-dependent fashion.

As used herein, the terms “modified” and “modification” when used in the context of the constituents of a nucleotide monomer, i.e., sugar, nucleobase and internucleoside linkage (backbone), refer to non-natural, changes to the chemical structure of these naturally occurring constituents or the substitutions of these constituents with non-naturally occurring ones, i.e., mimetics. For example, the “unmodified” or “naturally occurring” sugar ribose (RNA) can be modified by replacing the hydrogen at the 2′-position of ribose with a methyl group. See Monia, B. P. et al. J. Biol. Chem., 268: 14514-14522, 1993. Similarly, the naturally occurring internucleoside linkage is a 3′ to 5′ phosphodiester linkage that can be modified by replacing one of the non-bridging phosphate oxygen atoms with a sulfur atom to create a phosphorothioate linkage. See Geiser T. Ann N Y Acad Sci, 616: 173-183, 1990.

When used in the context of an oligonucleotide, “modified” or “modification” refers to an oligonucleotide that incorporates one or more modified sugar, nucleobase or internucleoside linkage. Modified oligonucleotides are structurally distinguishable, but functionally interchangeable with naturally occurring or synthetic unmodified oligonucleotides and usually have enhanced properties such as increased resistance to degradation by exonucleases and endonucleases, or increased binding affinity.

In some embodiments of the invention, the oligonucleotides comprise naturally-occurring nucleobases, sugars and covalent internucleoside linkages, i.e., those found in naturally occurring nucleic acids. In other embodiments, the oligonucleotides comprise non-naturally occurring, i.e., modified, nucleobases, sugars and/or covalent internucleoside linkages. In further embodiments, the oligonucleotides comprise a mixture of naturally occurring and non-naturally occurring nucleobases, sugars and/or covalent internucleoside linkages.

Non-naturally occurring internucleoside linkages “oligonucleotide backbones” include those that retain a phosphorus atom and also those that do not have a phosphorus atom. Numerous phosphorous containing modified oligonucleotide backbones are known in the art and include, for example, phosphoramidites, phosphorodiamidate morpholinos, phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, and phosphinates. In some embodiments, the modified oligonucleotide backbones are without phosphorus atoms and comprise short chain alkyl or cycloalkyl internucleoside linkages, mixed heteroatom and alkyl or cycloalkyl internucleoside linkages, or one or more short chain heteroatomic or heterocyclic internucleoside linkages. See Swayze E. and Bhat B. in Antisense Drug Technology Principles, Strategies, and Applications, 2nd Ed. CRC Press, Boca Rotan Fla., 2008 p. 144-182.

In further embodiments, the non-naturally occurring internucleoside linkages are uncharged and in others, the linkages are achiral. In some embodiments, the non-naturally occurring internucleoside linkages are uncharged and achiral, e.g., peptide nucleic acids (PNAs).

In some embodiments, the modified sugar moiety is a sugar other than ribose or deoxyribose. In particular embodiments, the sugar is arabinose, xylulose or hexose. In further embodiments, the sugar is substituted with one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modifications include 2′-methoxy (2′-O—CH3), 2′-aminopropoxy (2′-OCH2CH2CH2NH2), 2′-allyl (2′-CH2-CH═CH2), 2′-O-allyl (2′-O—CH2-CH═CH2) and 2′-fluoro (2′-F). The 2′-modification may be in the arabino (up) position or ribo (down) position. Similar modifications may also be made at other positions on an oligonucleotide, particularly the 3′ position of the sugar on the 3′ terminal nucleotide or in 2′-5′ linked oligonucleotides and the 5′ position of 5′ terminal nucleotide.

In some embodiments, the modified sugar is conformationally restricted. In further embodiments, the conformational restriction is the result of the sugar possessing a bicyclic moiety. In still further embodiments, the bicyclic moiety links the 2′-oxygen and the 3′ or 4′-carbon atoms. In some embodiments the linkage is a methylene (—CH2-)n group bridging the 2′ oxygen atom and the 4′ carbon atom, wherein n is 1 or 2. This type of structural arrangement produces what are known as “locked nucleic acids” (LNAs). See Koshkin et al. Tetrahedron, 54, 3607-3630, 1998; and Singh et al., Chem. Commun, 455-456, 1998.

In some embodiments, the modified sugar moiety is a sugar mimetic that comprises a morpholino ring. In further embodiments, the phosphodiester internucleoside linkage is replaced with an uncharged phosphorodiamidate linkage. See Summerton, Antisense Nucleic Acid Drug Dev., 7: 187-195, 1997.

In some embodiments, both the phosphate groups and the sugar moieties are replaced with a polyamide backbone comprising repeating N-(2-aminoethyl)-glycine units to which the nucleobases are attached via methylene carbonyl linkers. These constructs are called peptide nucleic acids (PNAs). PNAs are achiral, uncharged and because of the peptide bonds, are resistant to endo- and exonucleases. See Nielsen et al., Science, 1991, 254, 1497-1500 and U.S. Pat. No. 5,539,082.

Oligonucleotides useful in the methods of the invention include those comprising entirely or partially of naturally occurring nucleobases. Naturally occurring nucleobases include adenine, guanine, thymine, cytosine, uracil, 5-methylcytidine, pseudouridine, dihydrouridine, inosine, ribothymidine, 7-methylguanosine, hypoxanthine and xanthine.

Oligonucleotides further include those comprising entirely or partially of modified nucleobases (semi-synthetically or synthetically derived). Modified nucleobases include 5-methylcytosine (5-me-C), 5-hydroxymethyl cytosine, hypoxanthine, 2-aminoadenine, 2-methyladenine, 6-methyladenine, 2-propyladenine, N6-adenine, N6-isopentenyladenine, 2-methylthio-N6-isopentenyladenine, 2-methylguanine, 6-methylguanine, 2-propylguanine, 1-methylguanine, 7-methylguanine, 2,2-dimethylguanine, 2-thiouracil, 2-thiothymine and 2-thiocytosine, 5-fluorouracil, 5-bromouracil, 5-chlorouracil, 5-iodouracil, dihydrouracil, S-methyl-2-thiouracil, 2-thiouracil, 4-thiouracil, 5-methyluracil, uracil-5-oxyacetic acid methylester, uracil-5-oxyacetic acid, 5-carboxymethylaminomethyl-2-thiouridine, hypoxanthine, xantine, 4-acetylcytosine, 5-(carboxyhydroxylmethyl) uracil, 5-methoxycarboxymethyluracil, 5-methoxyuracil, 5-methyl-2-thiouracil, 3-(3-amino-3-N-2-carboxypropyl) uracil, 5-carboxymethylaminomethyluracil, 5-methylaminomethyluracil, 5-methoxyaminomethyl-2-thiouracil, 5-propynyl uracil and cytosine and other alkynyl derivatives of pyrimidine bases, 6-azo uracil, cytosine and thymine, 5-uracil (pseudouracil), 4-thiouracil, 8-halo-adenine, 8-amino adenine, 8-thiol adenine, 8-thioalkyl adenine, 8-hydroxyl adenine, 5-halo particularly 5-bromo, 5-trifluoromethyl uracil, 3-methylcytosine, 5-methylcytosine, 5-trifluoromethyl cytosine, 7-methylguanine and 7-methyladenine, 2-F-adenine, 2-amino-adenine, 8-azaguanine and 8-azaadenine, 7-deazaguanine, 8-halo-guanine, 8-amino guanine, 8-thiol guanine, 8-thioalkyl guanine, 8-hydroxyl guanine, 7-deazaadenine, 3-deazaguanine, 3-deazaadenine, beta-D-galactosylqueosine, beta-D-mannosylqueosine, inosine, l-methylinosine, 2,6-diaminopurine and queosine. Further modified nucleobases include tricyclic pyrimidines such as phenoxazine cytidine (1H-pyrimido[5,4-b][1,4]benzoxazin-2(3H)-one), and phenothiazine cytidine (1H-pyrimido[5,4-b][1,4]benzothiazin-2(3H)-one. See Herdewijn P, Antisense Nucleic Acid Drug Dev 10: 297-310, 2000; and Sanghvi Y S, et al. Nucleic Acids Res, 21: 3197-3203, 1993.

In some embodiments, at least one nucleoside, i.e., a joined base and sugar, in an oligonucleotide is modified, i.e., a nucleoside mimetic. In certain embodiments, the modified nucleoside comprises a tetrahydropyran nucleoside, wherein a substituted tetrahydropyran ring replaces the naturally occurring pentofuranose ring. See PCT/US2010/022759 and PCT/US2010/023397. In other embodiments, the nucleoside mimetic comprises a 5′-substituent and a 2′-substituent. See PCT/US2009/061913. In some embodiments, the nucleoside mimetic is a substituted α-L-bicyclic nucleoside. See PCT/US2009/058013. In additional embodiments, the nucleoside mimetic comprises a bicyclic sugar moiety. See PCT/US2009/039557. In further embodiments, the nucleoside mimetic comprises a bis modified bicyclic nucleoside. See PCT/US2009/066863. In certain embodiments, the nucleoside mimetic comprises a bicyclic cyclohexyl ring wherein one of the ring carbons is replaced with a heteroatom. See PCT/US2009/033373. In still further embodiments, a 3′ or 5′-terminal bicyclic nucleoside is attached covalently by a neutral internucleoside linkage to the oligonucleotide. See PCT/US2009/039438. In other embodiments, the nucleoside mimetic is a tricyclic nucleoside. See PCT/US2009037686.

Anti-CTGF oligonucleotides for use in the invention can contain any number of modifications described herein. In some embodiments, at least 5% of the nucleotides in the oligonucleotides are modified. In other embodiments, at least 10%, 15%, 20%, 30%, 35%, 40%, 45%, 50%, 55%, 60%, 65%, 70%, 75%, 80%, 85%, 90%, 95% or 99% of the nucleotides in the oligonucleotides are modified. In further embodiments, 100% of the nucleotides in the oligonucleotides are modified.

The aforementioned modifications may be incorporated uniformly across an entire oligonucleotide, at specific regions or discrete locations within the oligonucleotide including at a single nucleotide. Incorporating these modifications can create chimeric or hybrid oligonucleotides wherein two or more chemically distinct areas exist, each made up of one or more nucleotides.

Antisense oligonucleotides to CTGF useful in the methods of the invention include those disclosed in PCT/US2002/038618, PCT/US2009/054973 and PCT/US2009/054974; U.S. Pat. Nos. 6,358,741 and 6,965,025; and U.S. Provisional Patent Ser. No. 61/508,264. siRNA oligonucleotides to CTGF useful in the methods of the invention include U.S. Pat. Nos. 8,138,329, 7,622,454 and 7,666,853 and PCT/US2011/029849 and PCT/US2011/029867.

In some embodiments, the oligonucleotides further comprise a heterogeneous molecule covalently attached to the oligomer, with or without the use of a linker, also known as a crosslinker. In some embodiments, the heterogeneous molecule is a delivery or internalization moiety that enhances or assists the absorption, distribution and/or cellular uptake of the oligonucleotides. These moieties include polyethylene glycols, cholesterols, phospholipids, cell-penetrating peptides (CPPs) ligands to cell membrane receptors and antibodies. See Manoharan M. in Antisense Drug Technology: Principles. Strategies and Applications, Crooke S T, ed. Marcel Dekker, New York, N.Y., 2001, p. 391-470

Oligonucleotides useful in the methods of the invention may be conveniently and routinely made through the well-known technique of solid phase synthesis. Equipment for such synthesis is sold by several vendors including, for example, Life Technologies Corporation, Carlsbad, Calif. Any other means for such synthesis known in the art may alternatively be employed. Additionally, numerous service providers can be contracted to prepare the disclosed compounds.

### Methods

The present invention provides methods useful for treating peritoneal carcinomatosis. In one aspect of the invention, a method is provide for treating peritoneal carcinomatosis in a subject, the method comprising administering a therapeutically effective amount of an anti-CTGF agent to the subject. The methods of the present invention are applicable to all patients with peritoneal carcinomatosis regardless of whether the cancer originated in the peritoneum (primary) or whether arose in another organ or tissue (secondary). Applicable patients further include those with primary or secondary tumors in other locations in addition to peritoneal carcinomatosis, e.g., primary ovarian cancer in the pelvis and peritoneal carcinomatosis. Peritoneal carcinomatosis can be newly diagnosed, the result of refractory disease or recurrence following initial therapy or subsequent therapy.

Anti-CTGF agents can be administered using the disclosed methodologies as a neoadjuvant therapy administered before another therapy, such as immediately after diagnosis and before surgery or as adjuvant therapy in combination with other agents as front-line therapy, second-line therapy or salvage therapy. In some instances, the administration of an anti-CTGF agent can be used, alone or in combination with other therapeutic modalities to convert an otherwise, ineligible or borderline surgical candidate into a surgical candidate. Furthermore, the disclosed methodologies can be administered as maintenance therapy to maintain a complete response that was achieved by any means.

An administration route of particular interest is intraperitoneal (i.p.) administration as it would achieve high concentrations of an anti-CTGF agent within the peritoneal cavity. Additionally, i.p. administration will place the anti-CTGF agent in direct contact with individual cancer cells, micrometastases and tumors that adhere to or are invading into the peritoneum and hence are accessible to the i.p. instilled agent. In some embodiments, the anti-CTGF agent is co-administered by i.p. and i.v. administration, either sequentially or simultaneously. Since i.v. administered agents establish concentration gradients in tumors that decrease in concentration as the distance from the blood vessels increase, some tumor regions may not be exposed to optimal concentrations of a therapeutic agent By co-administering the anti-CTGF agents through i.p. and i.v. administration, more of areas within tumors, including the surface and areas close to the surface of the tumors, will be exposed to optimal therapeutic concentrations.

An anti-CTGF agent can be administered by i.p. administration as a neoadjuvant before cytoreductive surgery to induce apoptosis and inhibit the motility and adhesive ability of cancer cells that lie at the periphery of tumors and are most likely to be shed during surgery. In some embodiments, the anti-CTGF agent is administered i.p. at the time of a staging laparotomy. Additionally, an anti-CTGF agent can be administered during a surgical procedure, for example, cytoreductive surgery, including at the end of the procedure where the surgeon could wash all the exposed tissue surfaces with an anti-CTGF agent containing solution to ensure that any shed cancer cells, tumor fragments, micrometastases or solitary cancer cells remaining in the peritoneal cavity are exposed to the anti-CTGF agent. Alternately, the anti-CTGF agent could be administered with intraperitoneal hyperthermic chemotherapy or following interperitoneal hyperthermic chemotherapy as a last treatment before surgically closing the abdomen. The exposure of cancer cells to an anti-CTGF agent may further potentiate the cytotoxic effects of heat and chemotherapy with little or no additionally toxicity. In further embodiments, the anti-CTGF agent can be administered at any suitable time after surgery to treat shedded cancer cells, tumor fragments, micrometasteses or solitary cancer cells. In some embodiments, the surgeon will place an intraperitoneal access device during cytoreduction surgery to facilitate future i.p. administrations of the anti-CTGF agent. In other embodiments, the anti-CTGF agent can be administered i.p. at the time of a second or third look laparotomy.

### Therapeutic Agents

The methods of the present invention utilize anti-CTGF agents including anti-CTGF antibodies. Exemplary anti-CTGF antibodies for use in the methods of the present invention are described, e.g., in U.S. Pat. No. 5,408,040, PCT/US1998/016423, PCT/US1999/029652 and International Publication No. WO 99/33878. Preferably, the anti-CTGF antibody for use in the method is a monoclonal antibody. Preferably the antibody is a neutralizing antibody. In other preferred embodiments, the antibody is a human or humanized antibody to CTGF. In a more preferred embodiment, the antibody recognizes an epitope within domain 2 of human CTGF. Exemplary monoclonal anti-CTGF antibodies for use in the methods of the present invention include CLN1 or mAb1 described in U.S. Pat. No. 7,405,274. In a particular embodiment, the antibody is identical to CLN1, described in U.S. Pat. No. 7,405,274. In a specific embodiment, the antibody is the antibody produced by ATCC Accession No. PTA-6006 cell line, as described in U.S. Pat. No. 7,405,274. Variants of CLN1 that retain the binding and neutralization functions characteristic of CLN1 are also useful in the present invention. Such variants typically retain the variable regions of the heavy and/or light chain of the original neutralizing antibody, or minimally the complementarity determining regions (CDR) of heavy and light chains, and may contain substitutions and/or deletions in the amino acid sequences outside of those variable regions. Fragments and engineered versions of the original neutralizing antibody, e.g., Fab, F(ab)2, Fv, scFV, diabodies, triabodies, minibodies, nanobodies, chimeric antibodies, humanized antibodies, etc. are likewise useful in the method of the present invention as are antibody mimetics. Such antibodies, or fragments thereof can be administered by various means known to those skilled in the art. For example, antibodies are often injected intravenously, intraperitoneally, or subcutaneously.

The methods of the present invention further include anti-CTGF oligonucleotides. Exemplary anti-CTGF oligonucleotides for use in the methods of the present invention include antisense oligonucleotides to CTGF as disclosed in PCT/US2002/038618, PCT/US2009/054973 and PCT/US2009/054974; U.S. Pat. Nos. 6,358,741 and 6,965,025; and U.S. patent application Ser. No. 13/546,799. Additionally exemplary anti-CTGF oligonucleotides include CTGF siRNA oligonucleotides such as those disclosed in U.S. Pat. Nos. 8,138,329, 7,622,454 and 7,666,853; and PCT/US2011/029849 and PCT/US2011/029867.

In some embodiments, at least one additional therapeutic agent is administered. In further embodiments the additional therapeutic agent is a chemotherapy agent. As used herein, the term “chemotherapeutic agent” refers to any compound that can be used in the treatment, management or amelioration of cancer, including peritoneal carcinomatosis, or the amelioration or relief of one or more symptoms of a cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azascrine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycins, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′2″-trichlorotriethylamine; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; etoposide (VP-16); ifosfamide; mitomycin C; mitoxantrone; vinblastine; vincristine; vinorelbine; navelbine; novantrone; teniposide; daunomycin; aminopterin; xeloda; ibandronate; difluoromethylornithine (DMFO); retinoic acid; esperamicins; capecitabine; imexon; tyrosine kinase inhibitors, such as epidermal growth factor receptor tyrosine kinase inhibitor erlotinib; and pharmaceutically acceptable salts, acids or derivatives of any of the above.

In particular embodiments, the chemotherapeutic agent is capecitabine, carboplatin, cisplatin, cyclophosphamide, docetaxel, doxorubicin, epirubicin, erlotinib, 5-fluorouracil, gemcitabine, irinotecan, leucovorin, oxaliplatin, paclitaxel or topotecan. In some embodiments, the chemotherapy is administered as hyperthermic interperitoneal chemotherapy. In further embodiments, one or more chemotherapy agent is combined with concurrent radiotherapy. In particular embodiments, 5-fluorouracil is combined with concurrent radiotherapy.

In some embodiments, the additional therapeutic agent is an immunotherapy agent. Immunotherapy agent is defined broadly to include exogenously produced antibodies, such as bevacizumab, cetuximab, canitumumab or volociximab; vaccines, including, peptide vaccines, whole tumor cell vaccines, antigen-pulsed dendritic cell-based vaccines and DNA vaccines; and adoptive cell transfer.

In still further embodiments, the additional therapeutic agent is a genetic therapeutic agent selected from plasmids, naked DNA, transiently or stably transfected cells, antisense oligonucleotides and siRNA oligonucleotides.

In other embodiments, the additional therapeutic agent is surgery. In further embodiments, the surgery is debulking and/or cytoreductive surgery. Cytoreductive surgery attempts to completely remove tumor masses and may further include the resection of the greater omentum, right parietal peritonectomy, resection of right colon, left upper side and left parietal peritonectomy, splenectomy; right upper side peritonectomy, peritoneal stripping, diaphragm stripping, Glisson's capsule resection, Morrison pouch peritonectomy, lesser omentum resection, hepatic ileus cytoreduction, cholecystectomy, total or partial stomach resection, kidney resection, pelvic peritonectomy, sigmoid resection, hysterectomy and bilateral annexectomy; other bowel resections and bowel anastomosis.

In further embodiments, the additional therapeutic agent is radiation. The radiation can be administered as external beam x-rays or electrons. In specific embodiments, the external beam radiation is administered interoperatively. Radiation can also be administered internally, for example as a radiolabeled antibody, peptide, ligand, oligonucleotide or small molecule. Suitable radioisotopes for radiolabeling antibodies and other molecules include alpha particle emitters (e.g., ²²⁵Ac, ²¹¹At and ²¹³Bi), beta particle emitters (e.g., ¹³¹I and ⁹⁰Y) and Auger election emitters (e.g., ¹²³I, ¹²⁴I and ¹¹¹In). Typically, these types of radiolabeled molecules are soluble and can be administered by i.p. or i.v. administration. Alternately, the source of the internal radiation is insoluble or colloidal and can be administered through i.p. administration, for example phosphorus-32-labeled chromic hydroxide particles.

In some embodiments, combining an anti-CTGF agent with another therapeutic agent increases or potentiates the therapeutic efficacy of the other therapeutic agent with little or no additionally toxicity. In further embodiments, combining an anti-CTGF agent with another therapeutic agent increases the survival of the patient beyond what would be expected with the use of the other therapeutic agent alone. In other embodiments, combining an anti-CTGF agent with another therapeutic agent allows for the use of a lesser quantity, activity or dosage of the other therapeutic agent than is conventionally used, while maintaining or exceeding the other agent's expected therapeutic response at the higher, conventional quantity, activity or dosage. Further, the combination of an anti-CTGF agent with a lesser quantity, activity or dosage of the other therapeutic agent than is conventionally used, reduces the overall toxicity experienced by the patient as compared to the toxicity seen with the other therapeutic agent when used at the conventional dosage.

### Pharmaceutical Formulations

For therapeutic applications, anti-CTGF agents can be administered directly or formulated as pharmaceutical compositions. The anti-CTGF agents may be administered intravenously as a bolus or by continuous infusion over a period of time. Further, the anti-CTGF agents may be administered intraperitoneally. Alternately, the anti-CTGF agents may be administered by intramuscular, subcutaneous, intratumoral, peritumoral, oral, inhalation or topical mutes. The route of administration may influence the type and composition of formulation used in the anti-CTGF preparation.

Anti-CTGF agent formulations for use in accordance with the present invention may be prepared by mixing an anti-CTGF agent with pharmaceutically acceptable carriers, excipients or stabilizers that are nontoxic to recipients at the dosages and concentrations employed. Anti-CTGF agent formulations may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); carriers; hydrophilic polymers such as polyvinylpyrrolidone; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In particular, anti-CTGF antibody formulations may further comprise low molecular weight polypeptides; carriers such as serum albumin, gelatin, or immunoglobulins; and amino acids such as glycine, glutamine, asparagine, histidine, arginine, or lysine. The anti-CTGF antibody formulations can be lyophilized as described in PCT/US1996/012251.

Anti-CTGF oligonucleotides can be formulated as liposomes to increase drug accumulation at a target site, reduce drug toxicity and protect the encapsulated oligonucleotides in the internal compartments from metabolism and degradation. See Lian T. and Ho, R. J. Y. J Pharma Sci, 90: 667-680, 2001. Useful lipids for liposome construction include neutral lipids, e.g., dioleoylphosphatidyl ethanolamine and distearolyphosphatidyl choline; negative lipids, e.g., dimyristoylphosphatidyl glycerol and cationic lipids, e.g., dioleoylphosphatidyl ethanolamine dioleyloxypropyltrimethyl ammonium chloride.

Liposomes may incorporate glycolipids or be derivatized with one or more hydrophilic polymers, such as a polyethylene glycol (PEG) to enhance circulation lifetimes or peritoneal residence time relative to liposomes lacking such specialized lipids or hydrophilic polymers. See Uster P. S. et al. FEBS Letters, 1996, 386: 243-246. Additionally, liposomes can be targeted to specific cell types by coupling the liposome to antibodies, antibody fragments or ligands. See Yu B et al. Am Asso Pharma Sci, 11: 195-203, 2009.

Sustained-release preparations may also be prepared. Frequently, polymers such as poly(lactic acid), poly(glycolic acid), or copolymers thereof serve as controlled/sustained release matrices, in addition to others well known in the art. Numerous pharmaceutically acceptable carriers, excipients and stabilizers are available in the art, and include those listed in various pharmacopoeias, e.g., US Pharmacopeia, Japanese Pharmacopeia, European Pharmacopeia, and British Pharmacopeia. Other sources include the Inactive Ingredient Search database maintained by the FDA and the Handbook of Pharmaceutical Additives, ed. Ash; Synapse Information Resources, Inc. 3rd Ed. 2007.

Compositions formulated for parenteral administration by injection are usually sterile and, can be presented in unit dosage forms, e.g., in ampoules, syringes, injection pens, or in multi-dose containers, the latter usually containing a preservative. In certain instances, such as with a lyophilized product or a concentrate, the parenteral formulation would be reconstituted or diluted prior to administration. The formulations may also contain one or more chemotherapy agent as necessary for the particular indication being treated, preferably those with complementary activities that do not adversely affect each other. Various chemotherapy agents that can be combined with an anti-CTGF agent are described above. Such drugs are suitably present in combination in amounts that are effective for the treating peritoneal carcinomatosis.

### Prognosis of Ovarian Cancer

The methods of the invention further include methods for prognosing ovarian cancer and other CTGF-associated cancers such as pancreatic cancer. The methods comprise determining the percentage of tumor-associated fibroblasts in a carcinoma sample obtained from the subject that are positive for CTGF expression and comparing the percentage of CTGF positive tumor-associated fibroblasts in the sample to a reference percentage. The prognosis is then made based on whether the percentage of CTGF positive cells is above or below the reference percentage. Typically, patents that have a higher percentage of CTGF positive cells than the reference percentage have a more aggressive form of disease and also a worse prognosis. In some embodiments, with ovarian cancer, the reference percentage is about 50%, 60%, 70%, 75%, 80%, 85%, 90% or 95%. In particular embodiments, the reference percentage is about 90%

The level of expression of CTGF in tumor-associated fibroblasts can be based on protein expression or mRNA expression using any standard technique in the art including immunohistochemistry, in situ hybridization or the amplification of nucleic acids through methods such as polymerase chain reaction technology.

The methods of the invention further include a method for treating a subject with a CTGF-associated cancer such as ovarian cancer. A tumor sample is first obtained from the patient. This material can be from a biopsy, for example taken during a laparascopic examination, or from tumor excised during cytoreductive surgery. Then the percentage of tumor-associated fibroblasts that are positive for CTGF is determined and compared to a reference percentage. A treatment course is then selected based on the comparison. Typically, patients that have a greater percentage of CTGF positive tumor-associated fibroblasts than the reference percentage are treated more aggressively than patients that have a lesser percentage of CTGF positive tumor-associated fibroblasts than the reference percentage. This is because patients with a greater percentage of CTGF positive tumor-associated fibroblasts than the reference percentage generally have lower overall survival and more aggressive disease including more chemotherapy resistant disease.

### Articles of Manufacture

The present compositions may, if desired, be presented in a pack or dispenser device containing one or more unit dosage forms containing the anti-CTGF agent. Such a pack or device may, for example, comprise metal or plastic foil, such as a blister pack, glass and rubber stoppers, such as in vials, or syringes. The pack or device holds or contains an anti-CTGF agent composition that is effective for treating peritoneal carcinomatosis, including advanced ovarian cancer, and may have a sterile access port (for example the container may be an intravenous solution bag or a vial having a stopper pierceable by a hypodermic injection needle). The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Compositions comprising an anti-CTGF agent formulated in a compatible pharmaceutical carrier may be provided in an appropriate container that is labeled for treatment of a peritoneal carcinomatosis. The pack or dispenser device may be accompanied by a package insert that provides instructions for administering the anti-CTGF agent including specific guidance regarding dosing.

In a further embodiment, the article of manufacture further comprises a container comprising a second medicament, wherein the anti-CTGF agent is a first medicament. This article further comprises instructions on the package insert for treating the patient with the second medicament, in an effective amount.

The application also provides for kits used to prognose a subject with a CTGF-associated cancer such as ovarian cancer. The kits may also be used to select therapy for a subject with a CTGF-associated cancer by providing detection agents and reagents for the detection and/or quantification of CTGF mRNA or protein expression. Kits can also include instructions for interpreting the results obtained using the kit.

In some embodiments, the kits are oligonucleotide-based kits, which may comprise, for example: (1) an oligonucleotide, e.g., a detectably labeled oligonucleotide, which hybridizes to a nucleic acid sequence encoding CTGF or (2) a pair of primers useful for amplifying a CTGF nucleic acid molecule. Kits may also comprise, e.g., a buffering agent, a preservative, or a protein stabilizing agent. The kits can further comprise components necessary for detecting the detectable label (e.g., an enzyme or a substrate). The kits can also contain a control sample or a series of control samples which can be assayed and compared to the test sample. Each component of a kit can be enclosed within an individual container and all of the various containers can be within a single package, along with instructions for interpreting the results of the assays performed using the kit.

In some embodiments, the kits are antibody-based kits, which may comprise, for example: (1) a first antibody (e.g., attached to a solid support) which binds to CTGF; and, optionally, (2) a second, different antibody which binds to either CTGF or the first antibody and is conjugated to a detectable label.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Microarray Analysis of Primary HGSOC

### Tissue Specimens

Fifty-one primary HGSOC tumors were obtained as described (Bonomeu T, et al. Cancer Res 2005; 65:10602-12) from previously untreated ovarian cancer patients hospitalized at the Brigham and Women's Hospital between 1990 and 2000. Tumor classification was determined according to the International Federation of Gynecology and Obstetric (FIGO) staging system. Additionally, 10 normal ovaries were obtained as controls from patients that underwent surgery for unrelated gynecologic diseases. All specimens and their corresponding clinical information were collected under protocols approved by the institutional review boards of the institution.

### Microdissection, RNA Purification and Microarray Analysis

Microdissection and RNA isolation were performed as described (Bonome T, et al., supra). Briefly, fibroblasts from 7 μm frozen sections were microdissected using a MD LMD laser microdissecting microscope (Leica, Wetzlar, Germany). RNA was isolated immediately in RLT lysis buffer (Qiagen, Valencia, Calif.) and was extracted and purified using the RNeasy Micro kit (Qiagen, Valencia, Calif.). All purified total RNA specimens were quantified and checked for quality with a Bioanalyzer 2100 system (Agilent, Palo Alto, Calif.). Total RNA amplification and hybridization to Affymetrix UI33A 2.0 arrays (Affymetrix, Santa Clara, Calif.) were performed as described (Bonome T, et al., supra).

Microarray analysis was performed as described (Bonome T, et al., supra). Normalized data were uploaded into the NCI Microarray Analysis Database for quality-control screening and collation. BRB ArrayTools (version 3.5.0) software developed by Dr. Richard Simon and Amy Peng Lam (National Cancer Institute, Bethesda, Md.) was used to filter the array data and complete the statistical analysis.

### Gene Expression Data Analysis

To ensure that the samples used in this study were enriched for fibroblasts and not contaminated by other components of the tumor stroma, the gene expression profiles for the expression of markers of immune and endothelial cells were examined. Expression of the T-cell markers CD8 and CD45 and the endothelial cell markers TIE-2 and VEGFR1 were below the level of detection in most samples demonstrating that the samples were enriched for fibroblasts and not contaminated with other cell types.

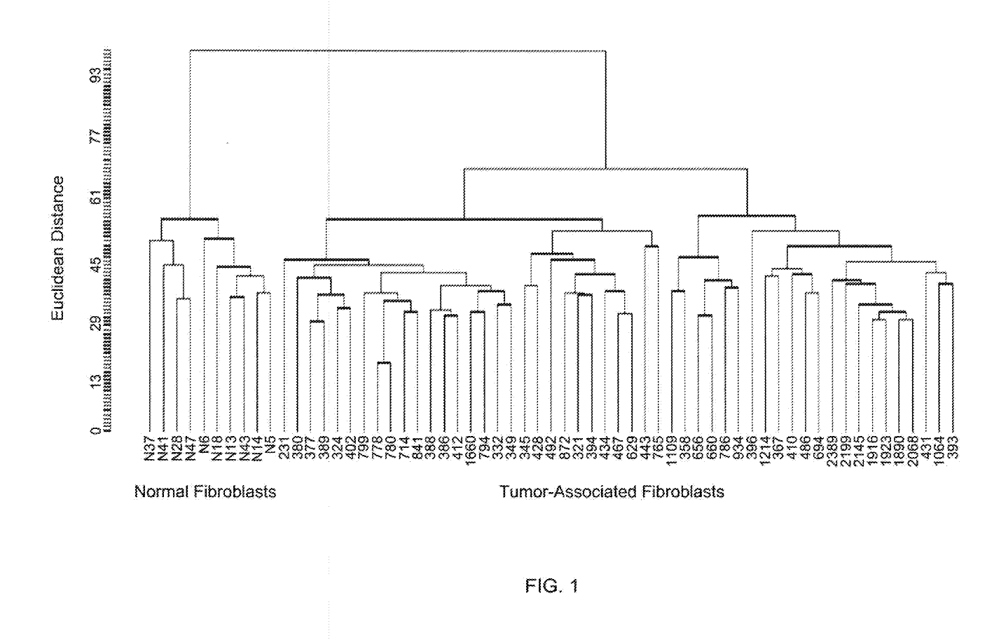

The microarray dataset was normalized and filtered to compare gene expression profiles in fibroblasts from normal ovaries to those from HGSOC tumors (tumor-associated fibroblasts). Filtering criteria identified 9,741 probe sets that were present in >50% of the arrays and whose expression was varied among the top 50^{th} percentile. An analysis of the probe sets by unsupervised hierarchical clustering of gene expression was performed using an Euclidean distance metric with average linkage to construct a dendogram to display associations between samples. This analysis clearly demonstrated that the normal ovarian fibroblasts and HGSOC-associated fibroblasts were markedly distinct from one another (FIG. 1). To identify those genes that significantly drove this distinction, all 9,741 probe sets were subjected to supervised class comparison analysis using a multivariate permutation test. A total of 2,703 probe sets, containing <10 false positives at a confidence of 95% corresponding, to 2,300 genes were identified as significantly differentially expressed between the HGSOC tumor-associated and normal fibroblast samples. Differential expression was considered significant at P<0.001. The differentially expressed genes were analyzed using PathwayStudio version 5.0 software (Ariadne Genomics, Rockville, Md.).

### Quantitative Real-Time PCR Validation

Nine genes differentially expressed between normal ovarian fibroblasts and HGSOC-associated fibroblasts were selected to validate the microarray results in all samples by qRT-PCR. CYR61, CTGF, SPP1 and TGFBR1 genes were selected because they are TGF-β-associated genes, while THBS1, MXRA5, LTBP2, RAB18 and COL11A1 were selected at random. Quantitative real-time PCR (qRT-PCR) was performed on 100 ng of double-amplified product from all patient samples using primer sets specific for 9 selected genes including CTGF and the housekeeping genes beta-glucuronidase (GUSB) and cyclophilin. An iCycler iQ Real-time PCR Detection System (Bio-Rad Laboratories, Hercules, Calif.) was used in conjunction with the SuperScript III Platinum SYBR Green One-Step qRT-PCR kit (Invitrogen, Carlsbad, Calif.) according to the manufacturer's instructions.

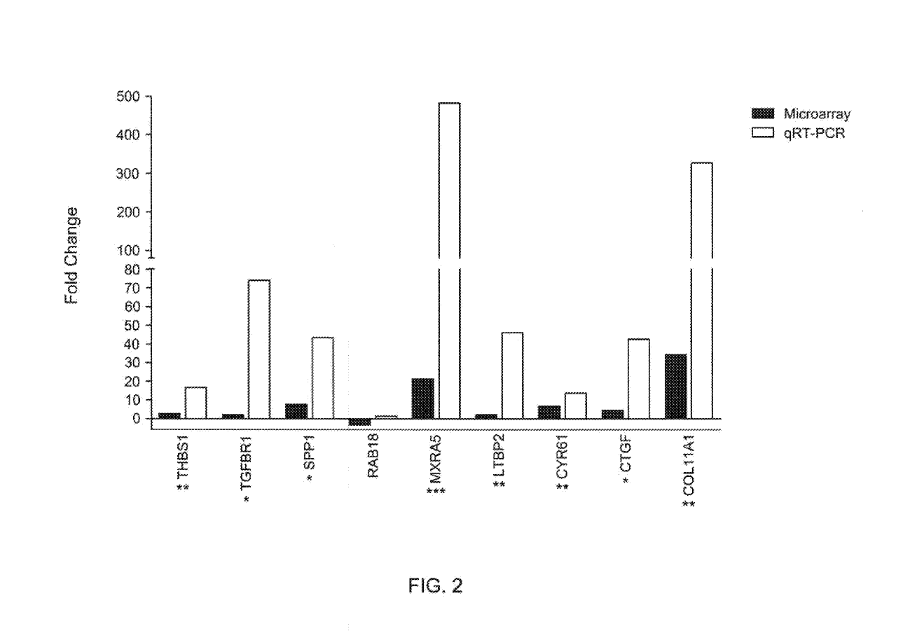

Of the 9 genes tested, 8 (THBS1, CYR61, CTGF, MXRA5, SPP1, LTBP2, TGFBR1 and COL11A1) were found to be significantly differentially expressed in HGSOC-associated fibroblasts, for a validation rate of 89%. The trends in gene expression levels across normal ovarian fibroblasts and HGSOC-associated fibroblast were consistent between qRT-PCR and microarray analysis (FIG. 2).

### Identification of Pathways Contributing to Role of Tumor-Associated Fibroblasts

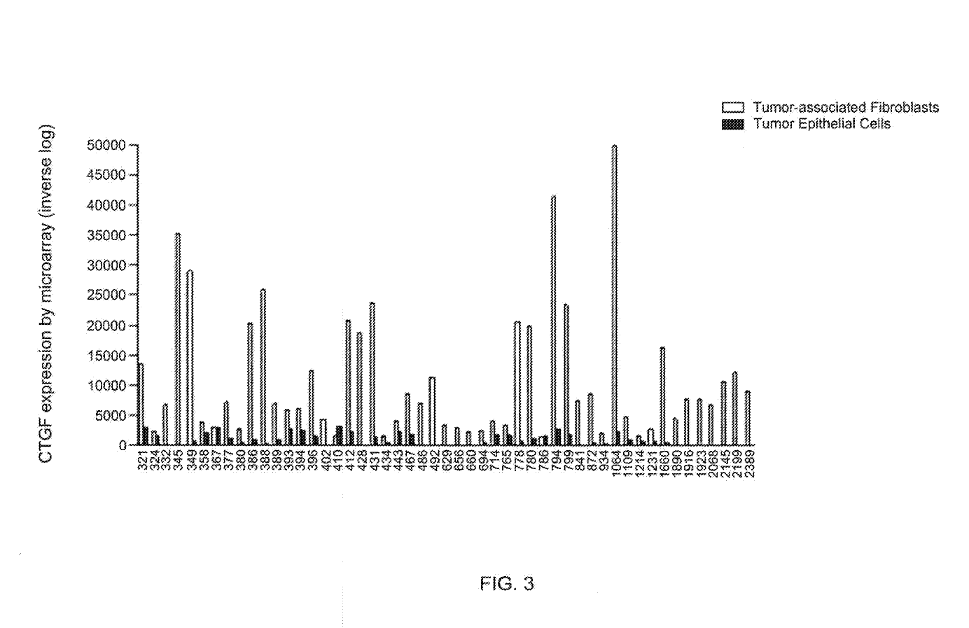

The PathwayStudio program was used to characterize the interactions between the 2,300 genes that were identified as differentially expressed in HGSOC-associated fibroblasts versus normal ovarian fibroblasts and to identify signaling pathways in the HGSOC-associated fibroblasts that may drive HGSOC progression. The expression of numerous genes in the TGF-β-regulated pathway was altered in HGSOC-associated fibroblasts (Table 1); the observed/expected ratio for genes in the “transforming growth factor beta receptor activity” ontology was 2.03 (p<0.001, Goeman's Test). These results suggest the role of the TGF-β pathway in stroma-driven tumor progression. The expression of CTGF is regulated by TGF-β and it was found to be differentially expressed between tumor-associated fibroblasts and matched tumor epithelial cells obtained from the same individuals. (FIG. 3) Interestingly, CTGF expression did not differ between normal ovary epithelial cells and ovarian fibroblasts.

TABLE 1
Expression of TGF-β-regulated genes* that are differentially
expressed between ovarian tumor-associated fibroblasts
and normal ovarian epithelial fibroblasts.

| Gene Symbol | Fold-change** | *P-value** |
|---|---|---|
| ACVR1 | 1.8 | 1.6E⁻⁰⁴ |
| ACVR1B | 2.1 | 8.0E⁻⁰⁶ |
| AHR | 2.7 | 1.4E⁻⁰⁵ |
| BCL2LI1 | −2.7 | 1.2E⁻⁰⁶ |
| BGN | 5.7 | 1.5E⁻⁰⁷ |
| Cd36 | −3.1 | 2.0E⁻⁰⁷ |
| CDK2AP1 | 4.2 | <1E⁻⁰⁷ |
| CFI | 3.9 | <1E⁻⁰⁷ |
| CLDN1 | −1.9 | 1.5E⁻⁰⁴ |
| COL1A2 | 5.0 | 1.4E⁻⁰⁵ |
| COL4A2 | 5.3 | <1E⁻⁰⁷ |
| CSPG2 | 9.0 | 1.4E⁻⁰⁶ |
| CTGF | 4.6 | 1.2E⁻⁰⁴ |
| CXCR4 | 10.8 | <1E⁻⁰⁷ |
| CYR61 | 6.7 | <1E⁻⁰⁷ |
| DCN | −3.4 | <1E⁻⁰⁷ |
| FGF2 | −2.6 | 7.4E⁻⁰⁶ |
| FN1 | 7.7 | <1E⁻⁰⁷ |
| ITGB5 | 3.3 | 5.7E⁻⁰⁶ |
| KPNA2 | 3.9 | <1E⁻⁰⁷ |
| LTBP2 | 3.1 | 4.2E⁻⁰⁵ |
| MAPK1 | 1.8 | 3.0E⁻⁰⁵ |
| PTEN | 2.7 | 5.2E⁻⁰⁵ |
| PTK2 | −3.3 | 1.5E⁻⁰⁷ |
| SMAD2 | 2.5 | <1E⁻⁰⁷ |
| SPP1 | 7.8 | 1.0E⁻⁰⁵ |
| TGFB1Il | 3.1 | <1E⁻⁰⁷ |
| TGFBR1 | 2.1 | 5.2E⁻⁰⁵ |
| TGFBR2 | 2.3 | 1.5E⁻⁰⁴ |
| TSC22 | −4.5 | 7.0E⁻⁰⁷ |
| VCL | 2.7 | <1E⁻⁰⁷ |
| YBX1 | 2.4 | 4.7E⁻⁰⁵ |

*Based on PathwayStudio ResNet database.
**For genes represented by multiple probe sets, the average fold-change and p-value is presented.

In some embodiments, a method is provided for treating peritoneal carcinomatosis, the method comprising reducing the mRNA expression or protein expression of genes whose expression is induced by TGF- or reducing the activity of proteins encoded by these genes. In further embodiments, a method is provided for treating peritoneal carcinomatosis comprising reducing the mRNA expression or protein expression of one or more of the following genes from Table 1 or the activity of the proteins encoded by these genes: activin A receptor, type 1 (ACVR1), activin receptor type-1B (ACVR1B), aryl hydrocarbon receptor (AHR), biglycan (BGN), cyclin-dependent kinase 2 associated protein 1 (CDK2AP1), complement factor 1 (CF1), collagen, type I, alpha 2 (COL1A2), collagen, type IV, alpha 2 (COL4A2), chondroitin sulfate proteoglycan 2 (CSPG2), connective tissue growth factor (CTGF), chemokine (C—X—C motif) receptor 4 (CXCR4), cysteine-rich, angiogenic inducer, 61 (CYR61), fibronectin 1 (FN1), integrin beta-5 (ITGB5), karyopherin alpha 2 (KPNA2), latent transforming growth factor beta binding protein 2 (LTBP2), mitogen-activated protein kinase 1 (MAPK1), phosphatase and tensin homolog (PTEN), SMAD family member 2 (SMAD2), secreted phosphoprotein 1 (SPP1), transforming growth factor beta 1 induced transcript 1 (TGFB1I1), transforming growth factor, beta receptor 1 (TGFBR1), transforming growth factor, beta receptor 1 (TGFBR2), vinculin (VCL) or Y box binding protein (YBX1). In further embodiments, the method for treating peritoneal carcinomatosis comprises reducing the mRNA expression, protein expression of one or more genes selected from the group consisting of ACVR1, CTGF, CXCR4, CYR61, ITGB5, TGF, TGFBR1 and TGFBR2. In other embodiments, the method for treating peritoneal carcinomatosis comprises reducing the activity of a protein encoded by a gene selected from the group consisting of ACVR1, CTGF, CXCR4, CYR61, ITGB5, TGF, TGFBR1 and TGFBR2. In particular embodiments, the gene is CTGF or CYR61.

In some embodiments, the treatment method reduces the mRNA or protein expression of one or more of the above identified genes from Table 1 by the use of antisense oligonucleotides or siRNA. In further embodiments, the treatment method reduces the activity of one or more proteins that are encoded by the above identified genes from Table 1. In some embodiments, the reduction in activity is achieved by the use of one or more antibodies that bind to the expressed proteins. In some embodiments, the antibodies are neutralizing antibodies. In other embodiments, the antibodies block the binding of the target molecule with a receptor, ligand, or cofactor. In particular embodiments, the reduction in protein activity is the reduction in CTGF activity. In further embodiments, the reduction in CTGF activity is achieved by the use of an anti-CTGF antibody. In specific embodiments, the anti-CTGF antibody is the antibody produced by the cell line identified by ATCC Accession No. PTA-6006.

In other embodiments, a method is provided for treating peritoneal carcinomatosis that comprises the reduction in gene expression, protein expression or protein activity of one or more genes in the TGF-β family or genes that encode receptors that bind TGF-β family members. In further embodiments, the reduction in gene expression, protein expression or protein activity is achieved by the use of antisense or siRNA to one or more genes within the TGF-β family or genes that encode for receptors of these TGF-β family members. In additional embodiments, the reduction in protein activity is achieved by the use of one or more antibodies to one or more TGF-β family members or receptors for these TGF-β family members.

In some embodiments, a method for treating peritoneal carcinomatosis is provided that comprises increasing the mRNA expression or protein expression of one of the following genes: BCL2-like 11 (BCL2L11), CD36 molecule (Cd36), claudin 1 (CLDN1), decorin (DCN), fibroblast growth factor 2 (FGF2), protein tyrosine kinase 2 (PTK2) and TGF-beta-stimulated clone-22 (TSC22). In other embodiments, the treatment method comprises the administration of exogenously produced BCL2L11, Cd36, CLDN1, DCN, FGF2, PTK2 or TSC22.

### Example 2

### Immunohistochemistry

To confirm the observed increased expression of CTGF in HGSOC-associated fibroblasts, immunohistochemical staining of CTGF was performed on 17 HGSOC tumors for which formalin-fixed paraffin-embedded tissue sections were available. Samples were de-paraffinized by incubating in xylene, rehydrated by soaking in 95% ethanol, followed by antigen retrieval in Target Retrieval Solution (DAKO, Carpinteria, Calif.) at 120° C. for 20 min. Slides were blocked in 3% hydrogen peroxide and sections were incubated with primary antibody (1:50 dilution) at room temperature for 60 min, washed two times with 1×TBS and incubated with horseradish peroxidase polymer for 30 min. Immunolocalization of CTGF protein was performed using a commercially available rabbit anti-CTGF polyclonal antibody, ab6992, (Abcam, Cambridge, Mass.) and the Picture MAX system (Zymed Laboratories Inc, Carlsbad, Calif.). CTGF positive signals were visualized using ACE Single Solution (Zymed Laboratories Inc, Carlsbad, Calif.). As a negative control, normal rabbit IgG was applied to HGSOC samples with high-levels of tumor-associated fibroblast CTGF expression. Tumor-associated fibroblast CTGF protein expression was quantified in one or two sections per case using Image-Pro Plus 5.1.0.20 for Windows (Media Cybernetics, Bethesda, Md.). The staining saturation was measured from 5 fixed-size areas in the stroma of both tumor and normal ovaries and averaged, yielding one score for each case.

Immunohistochemistry analysis demonstrated that CTGF protein expression was undetectable in the cortical stroma and the surface epithelium of normal ovary. In contrast, CTGF expression was significantly higher in HGSOC tumor stroma and was localized to tumor-associated fibroblasts. Further, the analysis showed that CTGF mRNA express and protein expression in the stroma was highly correlated (Pearsons r=0.636).

### Example 3

### TGF-β Regulation of CTGF

As CTGF is a TGF-β-regulated gene, the basal and TGF-β-stimulated levels of secreted CTGF were examined in the serous ovarian cancer cell line OVCAR3, as well as in normal and cancer-associated ovarian fibroblasts.

OVCAR3 cell line (American Type Culture Collection (ATCC, Manassas, Va.) was cultured in RPMI medium (Invitrogen, Carlsbad, Calif.) supplied with 10% fetal bovine serum and 20 mM L-glutamine and maintained in a humidified incubator at 37° and 5% CO₂. Normal ovarian fibroblasts (NF) and cancer-associated fibroblasts (CAF) were generously provided by Andrew Godwin (Fox Chase Cancer Center, Philadelphia, Pa.) and were validated by western blot to express vimentin and not keratin. Fibroblasts were maintained in DMEM medium (Invitrogen, Carlsbad, Calif.) supplied with 20% fetal bovine serum and 20 mM L-glutamine.

To test the ability of TGF-β to stimulate CTGF secretion, 10 ng/ml TGF-β (Peprotech, Rocky Hill, N.J.) and 50 μg/ml heparin (Sigma-Aldrich, St. Louis, Mo.) were added to cells in serum-free media and the cells incubated for 24 hrs. Secreted levels of CTGF in media were determined by a sandwich enzyme-linked immunosorbent assay (ELISA), using two distinct monoclonal antibodies against the CTGF protein (FibroGen, Inc., San Francisco, Calif.).

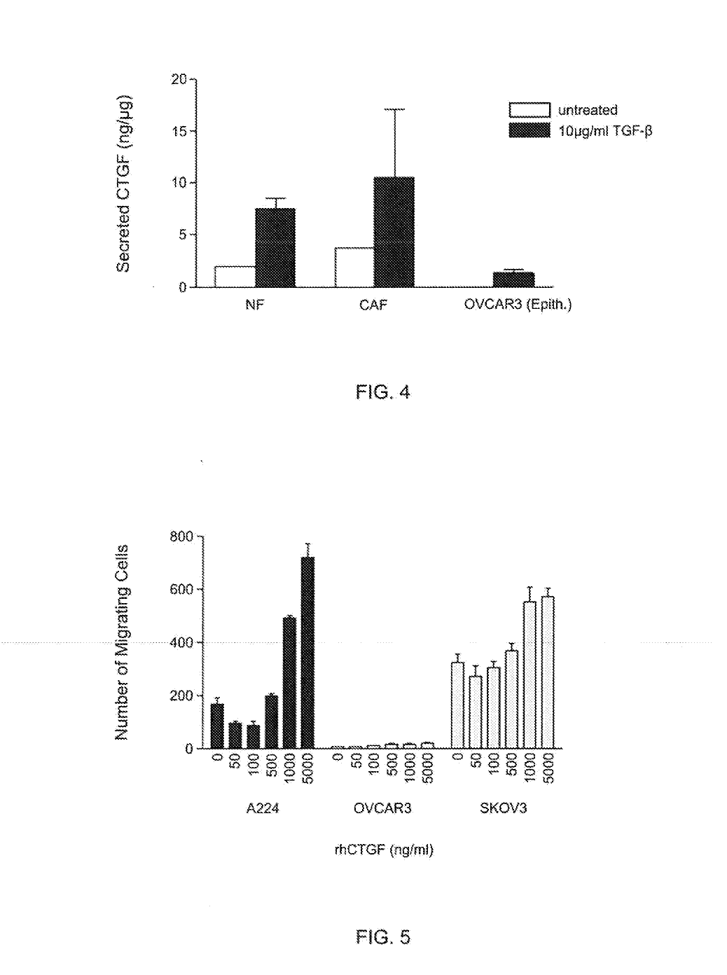

The basal level of secreted CTGF was undetectable in OVCAR3 cells (FIG. 4). This result is consistent with previous findings in HGSOC tumors. In contrast, both normal and cancer-associated ovarian fibroblasts secreted significantly higher levels of CTGF (p<0.05) with the cancer-associated ovarian fibroblasts exhibiting a 1.9-fold higher level of basal CTGF secretion compared to normal ovarian fibroblasts. (FIG. 4) Upon the addition of 10 μg/ml TGF-β μg/ml to the OVCAR3 cells, an extremely low level of secreted CTGF was detected. When 10 μg/ml TGF-β μg/ml was added to normal fibroblasts CTGF secretion increased 3.8-fold, while cancer-associated fibroblasts increased CTGF secretion by 2.8-fold. These results support the notion that the major source of CTGF in HGSOC tumors is the tumor-associated fibroblasts.

### Example 4

### Inhibition of CTGF Stimulated Tumor Cell Motility with an Anti-CTGF Antibody

To test whether CTGF stimulates ovarian cancer cell motility, CTGF was added to the media of three ovarian cancer cell lines that were in transwell migration chambers and the degree of migration measured. Briefly, A224 (ATCC), and SKOV3 cell lines (ATCC) and OVCAR3 cell lines were cultured in RPMI medium (Invitrogen, Carlsbad, Calif.) supplied with 10% fetal bovine serum and 20 mM L-glutamine and maintained in a humidified incubator at 37° and 5% CO₂. Cells were serum-starved overnight RPMI media/10% serum (500 μl) was added to lower wells of 8 micron PET membrane transwell culture chambers (BD Biosciences, San Jose, Calif.) and cells were seeded in 350 μl serum-free RPMI media in the upper wells. The ability of CTGF to stimulate cell motility was determined using recombinant human CTGF (5 μg/ml, FibroGen, Inc. San Francisco, Calif.). The ability of an anti-CTGF agent to block the expected CTGF-induced stimulation of cell motility was tested by adding either human anti-CTGF antibody, CLN1 (100 μg/ml, FibroGen, Inc. San Francisco, Calif.) or normal mouse IgG (100 μg/ml, Santa Cruz Biotech, Santa Cruz, Calif.) to the top and bottom wells. The culture chambers were then incubated at 37° C. for 6 hrs. The non-motile cells were removed from the upper surface of the membrane of each culture chamber with a cotton-tipped swab. The membranes were then fixed and stained using Diff-Quik stain (Dade Behring, Deerfield, Ill.). Three independent experiments were performed with triplicate samples. The number of migrating cells was calculated by counting the total number of cells in 5 fields at 20× magnification.

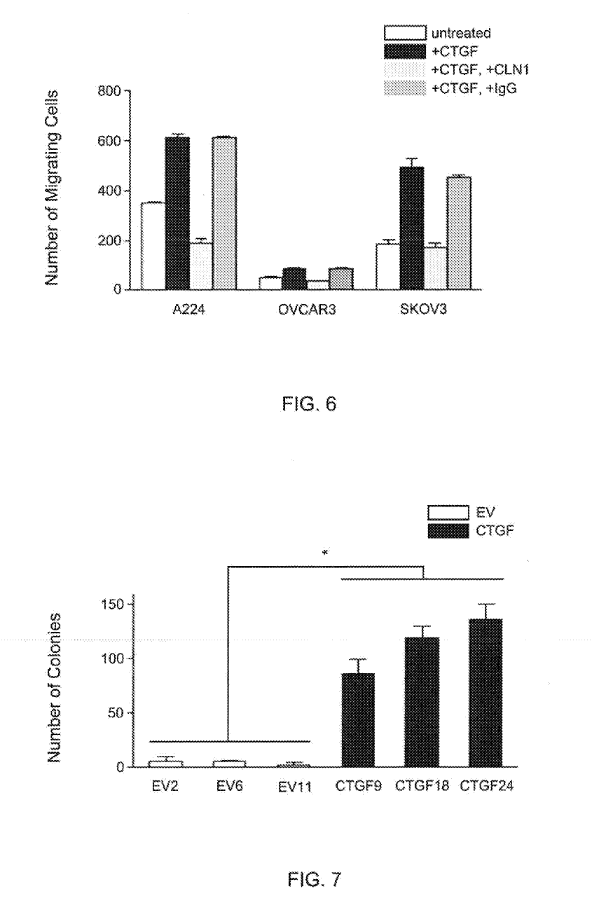

Recombinant human CTGF stimulated transwell migration of A224, OVCAR3 and SKOV3 cells in a dose-dependent manner (FIG. 5) (r=0.91, 0.68 and 0.78, respectively). The addition of 5 μg/ml rhCTGF for 6 hrs significantly stimulated migration of A224 (613±13.4 vs. 349±6.4 cells, p<0.008), OVCAR3 (88±2.1 vs. 51±3.5 cells, p<0.02) and SKOV3 (495±32.5 vs. 185±17.0 cells, p<0.02) (FIG. 6). The addition of 100 μg/ml CTGF-blocking antibody CLN1 significantly decreased transwell migration in the presence of recombinant CTGF in A224 (613±13.4 vs. 187±20.5 cells, p<0.004), OVCAR3 (88±2.1 vs. 37±1.4 cells, p<0.003) and SKOV3 (495±32.5 vs. 170±18.4 cells, p<0.02), while addition of IgG1 had no effect (FIG. 6).

### Example 5

### CTGF Stimulated Tumor Cell Proliferation

Recombinant human CTGF was tested for its ability to stimulate the cellular proliferation of A224, OVCAR3 and SKOV3 cell lines. Cell proliferation was measured using the CellTiter-Blue Cell Viability Assay (Promega, Madison, Wis.). In brief, 1000 cells were plated in 100 μl in 96-well plates. The next day, cells were serum-starved cells for 24 hr, followed by treatment with 5 μg/ml rhCTGF on day 1 and day 3. Each day. 20 μl of CellTiter-Blue reagent was added to each well. Following 3 hr incubation at 37° C., fluorescence was measured at an excitation wavelength of 560 nm and an emission wavelength of 590 nm. For each experiment, cells were plated in quadruplicate and the experiment was performed 3 independent times. Relative light units were calculated by subtracting the average background fluorescence (media only) from each well and averaging quadruplicate wells.

The addition of 5 μg/ml rhCTGF did not promote proliferation of any of the cell lines over a 5-day period. This lack of induced proliferation was reasoned to be due to the known instability of rhCTGF in culture media.

To overcome the suspected degradation of CTGF in media, several stably-transfected CTGF secreting cell lines were generated from OVCAR3 cells. In brief, the pcDNA3.1 vector containing HA-tagged CTGF (H. Phillip Koeffler, UCLA School of Medicine, Los Angeles, Calif.) or the empty pcDNA3 vector was transfected into OVCAR3 cells in 100 mm dishes using Effectene reagent (Qiagen, Valencia, Calif.) according to the manufacturer's instructions. Stable transfectants were selected and maintained in 300 μg/ml of G418. Following selection, 3 stably-transfected clones (clones 9, 18, 24) were produced by limited dilution cloning into 96-well plates. Over-expression of CTGF was confirmed by western blot, using an anti-CTGF antibody (clone L-20, Santa Cruz Biotechnology, Santa Cruz, Calif.) at 1:1000 dilution.

Anchorage independent growth of the stably-transfected cell lines was examined by soft agar cloning, three empty vector cell lines served as controls. In brief, a 7% stock of low-gelling agarose was diluted in RPMI media/10% serum to a final concentration of 0.7%. For the bottom layer of the plates, 1.5 mls of 0.7% agarose was added to 6-well plates and allowed to cool at 4° C. The leftover 0.7% agarose in media was further diluted in RPMI media/10% serum to a final concentration of 0.35%. For the top layer, 1000 cells were plated in 6 mls of 0.35% agarose. Following incubation for 1 hr at 4° C., the plates were transferred to 37° C. and incubated for ˜10-14 days. The cells were then stained overnight with 0.5 mg/ml of nitroblue tetrazolium (Sigma-Aldrich, St. Louis, Mo.) and colonies between 100-2000 microns were counted with the Biocount 4000P (Biosys, Germany). Two independent experiments were performed with triplicate samples.

All three CTGF over-expressing clones demonstrated significantly increased anchorage independent growth compared to empty vector controls (114±25.6 vs. 4±1.8 colonies, p<0.0001) (FIG. 7).

### Example 6

### Inhibition of Ex-Vivo Peritoneal Membrane Adhesion with an Anti-CTGF Antibody

To determine whether CTGF plays a role in peritoneal adhesion, an ex vivo assay modified from previous studies (Asao T, et al. Cancer Let. 1994; 78:57-62) was used. Briefly, peritoneal tissue was excised from euthanized 10-12 wk female Balb/c mice, divided along the midline into two pieces and placed into serum-free media. In 96-well plates, 100 μL of medium containing 5×10⁴ cells labeled with Syto9 green fluorescent nucleic acid stain (Life Technologies) was added to 100 μL of medium containing 5 μg/ml rhCTGF; 50 μg/ml CLN1; 125 μg/ml IgG; 5 μg/ml rhCTGF and 50 μg/ml CLN1; or 5 μg/ml rhCTGF and 125 μg/ml IgG. Peritoneal tissue was laid over the wells, mesothelial surface down, and then covered by a glass coverslip and the plate lid. The plates were incubated upside-down for 2 hrs at 37° C. The peritoneal tissue was then washed with serum-free medium, and attached cells observed and imaged using a Leica MZ16FA fluorescent dissection microscope, attached to a Leica DFC420C camera. Image J software (available from the National Institutes of Health website) was used to count 3 fields per well.

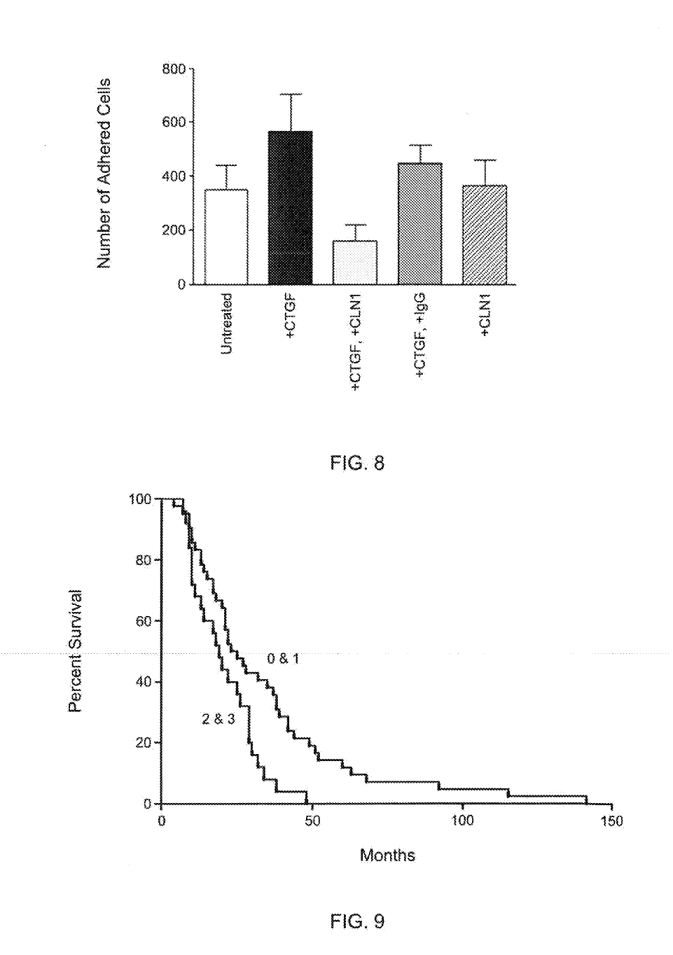

The addition of 5 μg/ml rhCTGF significantly increased adhesion of OVCAR3 (314±61.6 vs. 578±128.2 cells, p-value<2×10⁻⁶) to peritoneal tissue (FIG. 8). The addition of 50 μg/ml of the anti-CTGF antibody, CLN1, to rhCTGF significantly inhibited CTGF-mediated peritoneal adhesion (578±128.2 vs. 160±58.3 cells, p-value<2×10⁻⁸), while the addition of IgG1 had to rhCTGF no effect on CTGF-mediated peritoneal adhesion (FIG. 8).

### Example 7

### Anti-CTGF Antibody Treatment Reduces In Vivo Peritoneal Adhesions and Reduces Tumor Growth

Nude mice are inoculated with a human serous epithelial ovarian carcinoma derived cell line by i.p. administration. The mice are then randomized and divided into four groups. The first group receives i.p. administered anti-CTGF antibody immediately after tumor inoculation. The second group receives i.p. administered isotype matched murine IgG immediately after tumor inoculation as control. The third group receives i.p. administered anti-CTGF antibody 72 hours after tumor inoculation. The fourth group receives isotype matched murine IgG by i.p. administration 72 after tumor inoculation as control.

At 4 days and 1 week post-tumor inoculation, mice from each group are serial selected and sacrificed. Peritoneum tissue with any attached tumor cells including microscopic or macroscopic tumor nodules is removed. Tumor cells and tumor nodules are counted and then examined for the induction and degree of angiogenesis, apoptosis, proliferation, degree of invasion into the peritoneum, CTGF expression of tumor-associated fibroblasts and cell signaling.

The administration of an anti-CTGF antibody near the time of tumor inoculation greatly reduces the number of tumor cells that adhere to the peritoneum compared to isotype matched murine IgG treated mice. These results support the use of an anti-CTGF antibody following surgical excision of advanced ovarian cancer to reduce the incidence of recurrent peritoneal carcinomatosis due to surgically shed tumor cells.

The administration of an anti-CTGF antibody 72 hrs after tumor inoculation inhibits angiogenesis, induces apoptosis, retards proliferation and reduces tumor-associated fibroblast CTGF levels compared to isotype matched murine IgG treated animals. These results support the use of an anti-CTGF antibody for the treatment of established peritoneal carcinomatosis.

### Example 8

### Anti-CTGF Antibody Treatment Extends Survival in Peritoneal Carcinomatosis Model and is Synergistic with Chemotherapy

Nude mice are inoculated with a human serous epithelial ovarian carcinoma derived cell line by i.p. administration. The mice are then randomized and divided into four groups. Seven days following inoculation, the mice are treated. The first group receives i.p. administered isotype matched murine IgG as control. The second group receives i.p. administered anti-CTGF antibody. The third group receives i.p. administered cisplatin. The fourth group receives by i.p. administered anti-CTGF antibody and cisplatin.

The mice are followed for morbidity and mortality with mice in obvious distress euthanized. The isotype matched murine IgG treated mice have a median survival time of 22 days. The anti-CTGF antibody treated group has a median survival time of 28 days. The cisplatin treated group has a median survival time of 32 days. The combined anti-CTGF antibody and cisplatin treated group has a median survival time of 47 days. This experiment demonstrates the ability of an anti-CTGF agent to inhibit tumor growth and increase the survival of treated mice. The results of the combination treatment demonstrate the synergistic therapeutic effect achieved by the addition of anti-CTGF agent to a standard chemotherapy agent.

### Example 9

### High CTGF Expression Correlates with Lower Patient Survival

Tissue specimens (formalin-fixed, paraffin-embedded samples) were collected from patients undergoing primary laparotomy at the Gynecological Cancer Centre, Royal Hospital for Women, Sydney, Australia, following informed consent. Clinical, pathology and outcome data on each patient were collected and archived. All experimental procedures were approved by the Research Ethics Committee of the Sydney South East Area Hospital.

Archival tissue from 182 tumors removed at primary surgery (including endometrioid (n=12), mucinous (n=10), clear cell (n=13), serous (n=132), and other (n=4)) and 11 normal ovaries, removed during surgery for benign conditions, were included in the cohort.

Tissue core biopsies of 1.0 or 2.0 mm (n=614) were incorporated into medium-density tissue microarrays. Each patient was represented by two to five cores sampled from different areas of the tumor. Sections from each array were stained with H&E to confirm the inclusion of tumor tissue in each core, and cores containing no tumor were excluded from the study.

Four-μm sections were mounted on Superfrost Plus adhesion slides and heated in a convection oven at 75° C. for 2 h to promote adherence. Sections were dewaxed and rehydrated according to standard protocols, followed by an antigen unmasking procedure, using a high pH target retrieval solution (s2367; DAKO Australia Pty. Ltd., Campbellfield, Victoria, Australia). The primary anti-CTGF antibody (Fibrogen, San Francisco, Calif.) was used at 30 μg/ml. Bound antibody was detected using Novocastra NovoLink reagents (Leica Microsystems Pty. Ltd., North Ryde, New South Wales, Australia) and diaminobenzidine (DAKO) as a substrate. Negative controls used IgG (Cell Signaling Technology, Inc., Danvers, Mass.) as the primary antibody.

Counterstaining was performed with hematoxylin and 1% acid alcohol. Scoring of immunostaining was performed separately for epithelial cells and tumor-associated fibroblasts. Cores were scored for the intensity of staining (0-3) and the percentage of stained cells (0-100%). The highest intensity of tumor-associated fibroblast staining (0-3) seen across the cores for each patient was used as the score for comparing patient survival. In an alternate method, the survival analysis was performed using the total percentage of tumor-associated fibroblasts that stained positive for CTGF expression (0-100%).

Survival analyses was performed on individuals with stage 3 and 4 serous ovarian cancer whose follow up data indicated that they had died as a result of their malignancy. Patients were divided into low CTGF expression, i.e., a staining intensity score of 0 or 1; and high CTGF expression, i.e., a staining intensity score of 2 or 3. The length of survival was defined from the date of diagnosis to the date of patient death. There were 42 deaths in the low CTGF expression group and 25 deaths in the high CTGF expression group. The association between staining intensity and survival outcome was examined using a Kaplan-Meier analysis and a Cox proportional hazards model, and performed using Prism GraphPad. High CTGF expression in tumor-associated fibroblasts from serous ovarian cancer patients was associated with a lower median survival time, 19 months, compared to a median survival time of 24 months for patients with tumor-associated fibroblasts that had low CTGF expression. FIG. 9.

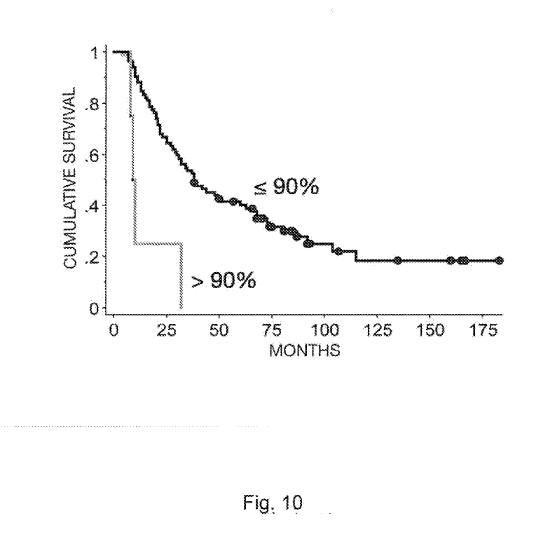

The comparison of the total percentage of tumor-associated fibroblasts that stained positive for CTGF demonstrated a direct correlation between increasing percentage of tumor-associated fibroblasts expressing CTGF and poorer overall survival. The greatest separation in overall survival was between cancer patients that had less than or equal to 90% CTGF positive tumor-associated fibroblasts (median overall survival of 38 months) and cancer patients that had greater than 90% CTGF positive tumor-associated fibroblasts (median overall survival of 9 months). FIG. 10. These results demonstrate that the level of CTGF expression in tumor-associated fibroblasts correlates with patient survival with higher CTGF expression scores associated with worse survival. Further, the results demonstrate that a patient's tumor-associated fibroblast CTGF expression level can be used as a prognostic indicator and that patients with high tumor-associated fibroblast CTGF expression levels should be selected for more aggressive treatment.

### Example 10

### Reduction of Peritoneal Carcinomatosis in Patient with Advanced Pancreatic Cancer

A patient with stage IIA pancreatic cancer undergoes surgery to remove the tumor and then receive conventional chemotherapy with gemcitabine. A complete response is achieved. A followup CT scan 8 months later detects scattered bilateral sub-5 mm pulmonary nodules and peritoneal carcinomatosis consisting of numerous scattered 5-10 mm peritoneal implants. The patient is administered a course of gemcitabine and an anti-CTGF antibody. Afterwards, the pulmonary nodules are not significantly changed in size, but a near complete resolution of the peritoneal carcinomatosis is achieved demonstrating the efficacy of an anti-CTGF antibody in combination with a chemotherapy agent in treating peritoneal carcinomatosis.

### Statistical Analysis

For validation of gene expression by quantitative real-time PCR, the relative expression for each gene was calculated using the 2^{-ΔΔCT} method, the CT values for the two housekeeping genes for a single reference gene value. The Goeman's Test was used to determine the significance of observed/expected ratios of differentially expressed genes within a gene ontology category. The Mann-Whitney U Test was used to compare medians of continuous variables between two independent samples in the immunohistochemistry study. R values indicate Pearson's correlation coefficients. For the in vitro studies, comparisons were made using two-tailed Student's t-test with the assumption of unequal variance and an alpha of 0.05.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1-20. (canceled)

21. A method of treating mesothelioma in a subject in need thereof, the method comprising administering to the subject an effective amount of an anti-connective tissue growth factor (CTGF) agent, thereby treating the mesothelioma.

22. The method of claim 21, wherein the anti-CTGF agent is an antibody, antibody fragment or antibody mimetic.

23. The method of claim 22, wherein the anti-CTGF agent is an antibody.

24. The method of claim 23, wherein the anti-CTGF antibody is identical to the antibody produced by the cell line identified by ATCC Accession No. PTA-6006.

25. The method of claim 21, wherein the anti-CTGF agent is an anti-CTGF oligonucleotide.

26. The method of claim 25, wherein the anti-CTGF oligonucleotide is an antisense oligonucleotide, siRNA, ribozyme or shRNA.

27. The method of claim 21, further comprising the administration of another therapeutic modality selected from the group consisting of chemotherapy, immunotherapy, gene therapy, surgery, radiotherapy, or hyperthermia.
